(19) United States
(12) Patent Application Publication
(10) Pub. No.: US 2005/0181016 A1
(43) Pub. Date: Aug. 18, 2005

(54) Decullularized extracellular matrix of conditioned body tissues and uses thereof

(76) Inventors: Toby Freyman, Waltham, MA (US); Wendy Naimark, Cambridge, MA (US); Maria Palasis, Wellesley, MA (US)

Correspondence Address: COOLEY GODWARD LLP;ATTN: PATENT GROUP, 11951 FREEDOM DRIVE, SUITE 1700, ONE FREEDOM SQUARE- RESTON TOWN CENTER, RESTON, VA, 20190-5061, US

(21) Appl. No.: 11/076,328

(22) Filed: Mar. 10, 2005

Related U.S. Application Data
(63) Continuation-in-part of application No. 10/622,293, filed on Jul. 17, 2003.

(51) Int. Cl. A61B 17/58

(52) U.S. Cl. 424/426; 606/104

(57) ABSTRACT

The invention is directed to an apparatus, such as a medical device, having a surface coated or covered with a decellularized extracellular matrix or having a component comprising the decellularized extracellular matrix for implantation into a subject, preferably a human. In one embodiment of the invention, a decellularized extracellular matrix is used to form a bodily implant such as a vein, an artery, an esophagus, or a ventricular restraining device. In some embodiments of the invention, the decellularized extracellular matrix is configured to be a time released therapeutic. In another embodiment of the invention, a decellularized extracellular matrix forms an aneurysm treatment device, such as an aneurysm coil, a seal, a pouch, or a filler. In a further embodiment of the invention, decellularized extracellular matrix is used to embolize lesions, tumors, or vessels. Methods for making the tissue regeneration scaffold and methods for manufacturing a coated or covered medical device having a component comprising decellularized extracellular matrix of body tissues are also provided.

### 1. CROSS-REFERENCES TO OTHER APPLICATIONS

This application is a continuation-in-part of U.S. patent application Ser. No. 10/622,293, entitled “Decellularized Extracellular Matrix of Conditioned Body Tissues and Uses Thereof,” filed on Jul. 17, 2003, and marked Attorney Docket No. 10177-118-999, the disclosure of which is incorporated herein by reference in its entirety.

### 2. FIELD OF THE INVENTION

The present invention relates generally to decellularized extracellular matrix of body tissues, including conditioned body tissues, as well as methods for the production and use thereof. In particular, the invention relates to treating defective, diseased, damaged or ischemic tissues or organs in a subject by injecting or implanting decellularized extracellular matrix of conditioned body tissue into a subject in need thereof. More particularly, the invention is directed to a tissue regeneration scaffold for implantation into a subject afflicted with a disease or condition that requires tissue or organ repair, regeneration and/or strengthening. Further, the invention is directed to a medical device, preferably a stent or an artificial heart, having a surface coated or covered with decellularized extracellular matrix from body tissue and/or having a component comprising the decellularized extracellular matrix for implantation into a subject, preferably a human. In one embodiment of the invention, a decellularized extracellular matrix is used to form a bodily implant such as a vein, an artery, an esophagus, or a ventricular restraining device. In some embodiments of the invention, the decellularized extracellular matrix is configured to be a time released therapeutic. In another embodiment of the invention, a decellularized extracellular matrix forms an aneurysm treatment device, such as an aneurysm coil, a seal, a pouch, or a filler. In a further embodiment of the invention, decellularized extracellular matrix is used to embolize lesions, tumors, or vessels. Methods for manufacturing a coated or covered medical device and methods for manufacturing a medical device having a component comprising decellularized extracellular matrix from body tissue and/or a coated or covered surface are also provided.

### 3. BACKGROUND OF THE INVENTION

Despite advances in medicine and healthcare, tissue and organ failure remain a frequent and costly occurrence. Each year in the United States, 40 to 90 million hospital days costing about $400 billion are attributed to the treatment of tissue and organ failure (Cohen et al., 1993, Chest 103(2):656). Incidents of cellular atrophy or injury to tissue and organ caused by trauma, burns, infection, inflammation, inadequate nutrition, diminished blood supply, loss of endocrine stimulation, aging, etc., are also prevalent. It is believed that a main pathway in the formation of cancer is considered to be repetitive tissue injury by highly chemically reactive free radicals and avid oxidants. Approximately eight million procedures are performed each year in the United States to treat patients suffering from tissue or organ injury or failure.

Traditionally, injured or diseased tissues or organs are treated by transplantation or through the use of a mechanical-type substitute. However, transplantation is associated with numerous complications (e.g., graft rejection, graft-versus-host disease) while mechanical substitutes only provide interim relief. Ultimately, the ideal treatments involve repairing or regenerating the tissue or organ. The application of functional genomics and developmental biology has accelerated tissue engineering product development by elucidating mechanisms of repair and regeneration. The use of animal products in the creation of tissue engineering products has provided important materials for the treatment, management or prevention of diseases or disorders that affect tissues and organs.

Soft tissue implantation represents an important step in tissue and organ healing. Soft tissue implants (as opposed to orthopedic, or hard tissue, implants), include biomaterials, synthetic materials, and tissues harvested from animals. The use of soft tissue implants are especially significant in the field of plastic and reconstructive surgery (Tarnow et al., 1996, J. Esthet. Dent. 8(1):12-9). For example, soft tissue implants can be used to reconstruct surgically or traumatically created tissue voids, to restore bulk to aging tissues, to correct soft tissue folds, and to augment tissue for cosmetic enhancement.

Diseased or damaged tendons, cartilage, and ligaments, on the other hand, are currently treated using orthopedic or hard tissue implants. Other treatment options include stimulation of bone marrow to form repair tissue, transplantation of osteochondral autografts or allografts, implantation of cultural autologous chondrocytes, and use of resorbable scaffolding (with or without cells).

In response to the need for more efficient and effective implant materials, the use of extracellular matrix (ECM) as templates for tissue or organ repair or regeneration has increased (Schmidt and Baier, 2000, Biomaterials 21:2215-31). Although the exact mechanisms through which ECM facilitates repair or regeneration are not known, the composition and the organization of the components are considered to be important factors that influence cell proliferation, gene expression patterns, and cell differentiation.

ECM is a complex structural entity surrounding and supporting cells. The extracellular matrix is found within mammalian tissues and is made up of three major classes of biomolecules: structural proteins (e.g., collagen and elastin), specialized proteins (e.g., fibrillin, fibronectin, and laminin), and proteoglycans (e.g., glycosaminoglycans). In addition to providing physical support to cells, the extracellular matrix affects cell function through mechanical and chemical signals.

Recent findings show porcine-derived, xenogeneic extracellular matrix derived from either the small intestinal submucosa or urinary bladder submucosa are useful as a tissue scaffold for esophageal repair in animal models (Badylak et al, 2000, J. Pediatr. Surg. 35(7):1097-10). Other studies have also shown that extracellular matrix derived from the submucosa of the porcine small intestine induces angiogenesis and host tissue remodelling when used as a xenogeneic bioscaffold in animal models of wound repair (Hodde et al, 2001, Endothelium 8(1):11-24). Cytokine analysis demonstrates that xenogeneic extracellular matrix grafts minimizes inflammatory response due to rejection (Allman et al, 2001, Transplantation 71(11):1631-40).

Despite current uses of extracellular matrix for tissue or organ repair or regeneration, it is often desirable that the extracellular matrix used for treatment contain an excess amount or a specific ratio of a particular protein, such as a growth hormone, preferably vascular endothelial growth factor (VEGF), to promote tissue growth, than that which naturally occurs in the extracellular matrix. There is a continued lack of suitable material that provides the best combination of biologically active materials and/or a desirable histoarchitecture as an implant to repair, regenerate or strengthen tissue or organs. There has yet to be developed a completely biocompatible, long-lasting implant that promotes and/or expedites tissue or organ repair or regeneration. Hence, the goal of the present invention is to provide body implants that are engineered for a specific application for a specific tissue or organ (i.e., an implant that provides a specific composition of biologically active material and mechanical properties).

The invention also relates to using a decellularized extracellular matrix to form a bodily implant such as a vein, an artery, an esophagus, or a ventricular restraining device. For example, the decellularized extracellular matrix may be formed into a tubular member. The tubular member may then be implanted into a body of a patient. In some embodiments of the invention, the decellularized extracellular matrix is configured to be a time released therapeutic.

The invention further relates to forming an aneurysm treatment device, such as an aneurysm coil, a seal, a pouch, or a filler, from a decellularized extracellular matrix. The invention also relates to using decellularized extracellular matrix to embolize lesions, tumors, or vessels.

### 4. SUMMARY OF THE INVENTION

To achieve the aforementioned objectives, we have invented an injectable or implantable composition comprising decellularized extracellular matrix obtained from conditioned body tissue of a donor subject. In particular, the invention relates to methods for producing the decellularized extracellular matrix by conditioning body tissue from a donor animal to produce a biological material, allowing the conditioned body tissue to produce the biological material, harvesting the conditioned body tissue from the donor animal, and decellularizing the harvested and conditioned body tissue to obtain the extracellular matrix containing the biological material.

In certain embodiments, the body tissue is conditioned in vivo or in situ before being harvested. In certain other embodiments, the body tissue is conditioned in vitro after being harvested. If the body tissue is conditioned in vivo or in situ, conditioning may be performed locally or systemically. If the body tissue is conditioned in vitro, conditioning may be performed in a bioreactor. The conditioned body tissue is given a period of time before and/or after harvest to produce the biological material in an amount of interest. The amount of biological material produced by the body tissue may be monitored before, during or after the conditioning step.

The body tissue may be conditioned using any one or more biological, chemical, pharmaceutical, physiological and/or mechanical treatment(s). In one embodiment, the body tissue is biologically conditioned by transfecting the body tissue with a nucleic acid. In another embodiment, the body tissue is chemically conditioned by incubating the body tissue in a hypotonic or hypertonic solution. In yet another embodiment, the body tissue is pharmaceutically conditioned by delivering a therapeutic agent to the body tissue. In yet another embodiment, the body tissue is physiologically conditioned by exposing the body tissue to heat shock. In yet another embodiment, the body tissue is mechanically conditioned by applying a force to the body tissue. Preferably, the force is produced by the expansion of a balloon against the body tissue.

The body tissue from a donor subject may be conditioned so that the biochemical composition and histoarchitecture of the body tissue is retained. In certain embodiments, the body tissue may be conditioned so that the biochemical composition and histoarchitecture of the body tissue from the donor subject is similar to the body tissue that is being repair, replaced and/or regenerated in a recipient subject. The body tissue may be from a mammal, preferably a pig or human.

The conditioned body tissue may retain or possess new physical properties such as strength, resiliency, density, insolubility, and permeability as compared to the unconditioned body tissue. The conditioned body tissue may also contain a biological material in an amount different than the amount of the biological material that the body tissue would produce absent the conditioning. In a specific embodiment, the biological material is a growth factor, preferably vascular endothelial growth factor (VEGF). In another specific embodiment, the biological material is an extracellular matrix protein, preferably elastin.

The harvested and conditioned body tissue may be decellularized using a combination of physical, chemical, and biological processes. The methods of the present invention involve the steps of decellularization by removing native cells, antigens, and cellular debris from the extracellular matrix of the body tissue. Preferably, an enzyme treating step is involved.

The body tissue may be further processed after decellularization to facilitate administration, injection or implantation. For examples, the decellularized extracellular matrix can be dried, concentrated, diluted, lyophilized, cryopreserved, electrically charged, sterilized, etc. In a preferred embodiment, the decellularized extracellular matrix is suspended in a saline solution as a final product.

The invention also relates to the administration, injection or implantation of the decellularized extracellular matrix of conditioned body tissue into a subject in need thereof. The decellularized extracellular matrix of the invention may be administered, injected or implanted alone or in combination with other therapeutically or prophylactically effective agents useful for treating, managing or preventing a disease or condition that requires tissue or organ repair, restoration and/or strengthening may be delivered to the body tissue before and/or after conditioning and/or harvesting.

The decellularized extracellular matrix may also be administered, injected or implanted before, during or after treatment with other methods of repairing, regenerating and/or strengthening of the diseased, defected, damaged or ischemic tissue or organ. In particular, the decellularized extracellular matrix may be used to promote angiogenesis and/or repair, replace or regenerate cells, tissues or organs, such as but not limited to lymph vessels, blood vessels, heart valves, myocardium, pericardium, pericardial sac, dura mater, meniscus, omentum, mesentery, conjunctiva, umbilical cords, bone marrow, bone pieces, ligaments, tendon, tooth implants, dermis, skin, muscle, nerves, spinal cord, pancreas, gut, intestines, peritoneum, submucosa, stomach, liver, and bladder.

The decellularized extracellular matrix of the present invention can also be used to form a tissue regeneration scaffold for implantation into a subject. The tissue regeneration scaffold may be used as a therapeutic to treat diseases or conditions that may benefit from improved angiogenesis, cell proliferation and/or tissue regeneration and/or strengthening. Such diseases or conditions include but are not limited to, bums, ulcer, trauma, wound, bone fracture, diabetes, psoriasis, arthritis, asthma, cystitis, inflammation, infection, ischemia, restenosis, stricture, atherosclerosis, occlusion, stroke, infarct, aneurysm, abdominal aortic aneurysm, uterine fibroid, urinary incontinence, vascular disorders, hemophilia, cancer, and organ failure (e.g., heart, kidney, lung, liver, intestine, etc.).

The invention further relates to a medical device comprising decellularized extracellular matrix of conditioned body tissue and methods for manufacturing such a medical device. The medical device is suitable for insertion into a subject, preferably a human. Preferably, the medical device is non-biodegradable. More preferably, the medical device is a stent or an artificial heart. In a specific embodiment, the decellularized extracellular matrix is coated onto the medical device. Preferably, the decellularized extracellular matrix is coated onto the medical device by spray coating, with or without a polymer carrier, or dip coating. In another specific embodiment, the decellularized extracellular matrix is used to construct a component of the medical device such as a wired-like elements of a stent or a valve of an artificial heart. The decellularized extracellular matrix may be used alone or in combination with a bulk polymer or biologically active material, preferably paclitaxel, to make, cover or coat the medical device.

4.1 Definitions

As used herein, the term “therapeutically effective amount” refers to that amount of the therapeutic agent sufficient to treat or manage defective, diseased, damaged or ischemic tissues or organs. A therapeutically effective amount may refer to the amount of therapeutic agent sufficient to delay or minimize the onset of symptoms associated with defective, diseased, damaged or ischemic tissues or organs. A therapeutically effective amount may also refer to the amount of the therapeutic agent that provides a therapeutic benefit in the treatment or management of the defective, diseased, damaged or ischemic tissues or organs. Further, a therapeutically effective amount with respect to a therapeutic agent of the invention means that amount of therapeutic agent alone, or in combination with other agents or therapies, that provides a therapeutic benefit in the treatment or management of defective, diseased, damaged or ischemic tissues or organs. Used in connection with an amount of the decellularized extracellular matrix of the invention, the term can encompass an amount that improves overall therapy, reduces or avoids unwanted effects, or enhances the therapeutic efficacy of or synergies with another therapeutic agent.

As used herein, the term “prophylactically effective amount” refers to that amount of the prophylactic agent sufficient to result in the prevention of the occurrence of defective, diseased, damaged or ischemic tissues or organs. A prophylactically effective amount may also refer to the amount of prophylactic agent sufficient to prevent the occurrence or recurrence of defective, diseased, damaged or ischemic tissues or organs in a patient, including but not limited to those (genetically) predisposed. A prophylactically effective amount may also refer to the amount of the prophylactic agent that provides a prophylactic benefit in the prevention of defective, diseased, damaged or ischemic tissues or organs. Further, a prophylactically effective amount with respect to a prophylactic agent of the invention means that amount of prophylactic agent alone, or in combination with other agents or therapies, that provides a prophylactic benefit in the prevention of the occurrence or recurrence of defective, diseased, damaged or ischemic tissues or organs. Used in connection with an amount of the decellularized extracellular matrix of the invention, the term can encompass an amount that improves overall prophylaxis or enhances the prophylactic efficacy of or synergies with another prophylactic agent.

As used herein, the terms “subject” and “patient” are used interchangeably. As used herein, a subject is preferably a mammal such as a non-primate (e.g., cows, pigs, horses, cats, dogs, rats, etc.) and a primate (e.g., monkey and human), most preferably a human.

As used herein, the term “body tissue” broadly encompasses any or a number of cells, tissues or organs.

As used herein, the term “repair” relates to the restoration of defective, diseased, damaged or ischemic tissues or organs to a sound or healthy stage by replacing a part or putting together what is defective, diseased, damaged or ischemic by synthesizing and incorporating additional normal cells, tissue or organ components to increase the size and/or strength of the defective, diseased, damaged or ischemic tissue or organ.

As used herein, the term “replace” relates to the substitution of defective, diseased, damaged or ischemic tissues or organs with newly synthesized cells, tissue or organ components facilitated by the decellularized extracellular matrix of the present invention.

As used herein, the term “regenerate” relates to the regrowth and/or reconstitution of defective, diseased, damaged or ischemic tissues or organs.

As used herein, the term “strengthen” relates to the making stronger of the defective, diseased, damaged or ischemic tissues or organs.

As used herein, the terms “biological material” and “biologically active material” are used interchangeably. Examples of a biological material include, but are not limited to, vascular endothelial growth factor (VEGF), transforming growth factor (TGF), fibroblast growth factor (FGF), epidermal growth factor (EGF), cartilage growth factor (CGF), nerve growth factor (NGF), keratinocyte growth factor (KGF), skeletal growth factor (SGF), osteoblast-derived growth factor (BDGF), hepatocyte growth factor (HGF), insulin-like growth factor (IGF), cytokine growth factors (CGF), platelet-derived growth factor (PDGF), hypoxia inducible factor-1 (HIF-1), stem cell derived factor (SDF), stem cell factor (SCF), endothelial cell growth supplement (ECGS), granulocyte macrophage colony stimulating factor (GM-CSF), growth differentiation factor (GDF), integrin modulating factor (IMF), calmodulin (CaM), thymidine kinase (TK), tumor necrosis factor (TNF), growth hormone (GH), bone morphogenic proteins (BMP), matrix metalloproteinase (MMP), tissue inhibitor of matrix metalloproteinase (TIMP), cytokines, interleukins, lymphokines, interferon, integrin, collagen (all types), elastin, fibrillins, fibronectin, laminin, glycosaminoglycans, vitronectin, proteoglycans, transferrin, cytotactin, cell binding domains (e.g., RGD), and tenascin.

As used herein, the term “analog” refers to a polypeptide that possesses a similar or identical function as a particular protein (e.g., vascular endothelial growth factor), or a fragment thereof, but does not necessarily comprise a similar or identical amino acid sequence or structure of that protein or a fragment thereof. A polypeptide that has a similar amino acid sequence refers to a polypeptide that satisfies at least one of the following: (a) a polypeptide having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of a protein or a fragment thereof as described herein; (b) a polypeptide encoded by a nucleotide sequence that hybridizes under stringent conditions to a nucleotide sequence encoding a protein or a fragment thereof as described herein of at least 20 amino acid residues, at least 30 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues; and (c) a polypeptide encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the nucleotide sequence encoding a protein or a fragment thereof as described herein. A polypeptide with similar structure to a protein or a fragment thereof as described herein refers to a polypeptide that has a similar secondary, tertiary or quaternary structure of a protein or a fragment thereof as described herein. The structure of a polypeptide can be determined by methods known to those skilled in the art, including but not limited to, X-ray crystallography, nuclear magnetic resonance, and crystallographic electron microscopy.

As used herein, the term “derivative” refers to a polypeptide that comprises an amino acid sequence of a protein, such as vascular endothelial growth factor, a fragment of the protein, an antibody that immunospecifically binds to the protein, or an antibody fragment that immunospecifically binds to the protein which has been altered by the introduction of amino acid residue substitutions, deletions or additions. The term “derivative” as used herein also refers to the protein, a fragment of the protein, an antibody that immunospecifically binds to the protein, or an antibody fragment that immunospecifically binds to the protein which has been modified, i.e., by the covalent attachment of any type of molecule to the polypeptide. For example, but not by way of limitation, by glycosylation, acetylation, pegylation, phosphorylation, amidation, derivatization by known protecting/blocking groups, proteolytic cleavage, linkage to a cellular ligand or other protein, etc. A derivative may also be modified by chemical modifications using techniques known to those of skill in the art, including, but not limited to specific chemical cleavage, acetylation, formylation, metabolic synthesis of tunicamycin, etc. Further, the derivative may contain one or more non-classical amino acids. In one embodiment, the derivative possesses a similar or identical function as the protein of interest. In another embodiment, the derivative has an altered activity when compared to an unaltered protein. For example, a derivative antibody or fragment thereof can bind to its epitope more tightly or be more resistant to proteolysis.

As used herein, the term “fragment” refers to a peptide or polypeptide comprising an amino acid sequence of at least 20 contiguous amino acid residues, at least 30 contiguous amino acid residues, at least 40 contiguous amino acid residues, at least 50 contiguous amino acid residues, at least 60 contiguous amino residues, at least 70 contiguous amino acid residues, at least contiguous 80 amino acid residues, at least contiguous 90 amino acid residues, at least contiguous 100 amino acid residues, at least contiguous 125 amino acid residues, at least 150 contiguous amino acid residues, at least contiguous 175 amino acid residues, at least contiguous 200 amino acid residues, or at least contiguous 250 amino acid residues of the amino acid sequence of a protein, such as vascular endothelial growth factor.

The percent identity of two amino acid sequences or of two nucleic acid sequences is determined by aligning the sequences for optimal comparison purposes (e.g., gaps can be introduced in the first sequence for best alignment with the sequence) and comparing the amino acid residues or nucleotides at corresponding positions. The “best alignment” is an alignment of two sequences which results in the highest percent identity. The percent identity is determined by the number of identical amino acid residues or nucleotides in the sequences being compared (i.e., % identity=# of identical positions/total # of positions×100).

The determination of percent identity between two sequences can be accomplished using a mathematical algorithm known to those of skill in the art. An example of a mathematical algorithm for comparing two sequences is the algorithm of Karlin and Altschul, 1990, Proc. Natl. Acad. Sci. USA 87:2264-2268, modified as in Karlin and Altschul, 1993, Proc. Natl. Acad. Sci. USA 90:5873-5877. The NBLAST and XBLAST programs of Altschul et al., 1990, J. Mol. Biol. 215:403-410 have incorporated such an algorithm. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al., 1997, Nucleic Acids Res. 25:3389-3402. Alternatively, PSI-Blast can be used to perform an iterated search which detects distant relationships between molecules (Id.). When utilizing BLAST, Gapped BLAST, and PSI-Blast programs, the default parameters of the respective programs (e.g., XBLAST and NBLAST) can be used. See http://www.ncbi.nlm.nih.gov.

Another example of a mathematical algorithm utilized for the comparison of sequences is the algorithm of Myers and Miller, CABIOS (1989). The ALIGN program (version 2.0) which is part of the CGC sequence alignment software package has incorporated such an algorithm. Other algorithms for sequence analysis known in the art include ADVANCE and ADAM as described in Torellis and Robotti, 1994, Comput. Appl. Biosci. 10:3-5; and FASTA described in Pearson and Lipman, 1988, Proc. Natl. Acad. Sci. 85:2444-8. Within FASTA, ktup is a control option that sets the sensitivity and speed of the search.

### 5. DETAILED DESCRIPTION OF THE INVENTION

The present invention relates to decellularized extracellular matrix of body tissue, including conditioned body tissue. In certain embodiments, the body tissue of a donor subject is conditioned in vivo or in situ before harvest. In certain embodiments, the body tissue of a donor subject is first harvested and then conditioned in vitro, preferably in a bioreactor. The conditioned body tissue is given a period of time to produce a biological material in an amount different than the amount that is produced by a body tissue absent the conditioning. The conditioned body tissue may be decellularized by at least one or a combination of physical, chemical and/or biological step(s). Preferably, the decellularized conditioned body tissue is rid of cellular components and only retains the extracellular matrix and the biological material of interest. In certain embodiments, the decellularized conditioned body tissue can be further processed prior to its use.

The decellularized extracellular matrix may be grafted directly onto the site of a defective, diseased, damaged or ischemic tissue or organ. The decellularized extracellular matrix may also be processed into a formulation and injected at a site in need of treatment. The decellularized extracellular matrix may further be used in a tissue regeneration scaffold for implantation into a subject. In addition, the decellularized extracellular matrix can be part of a medical device, preferably a stent, an artificial heart, a ventricular restraining device, or an aneurysm coil for implantation into a subject. For instance, the decellularized extracellular matrix can be coated onto the medical device, preferably by spray coating or dip coating, or incorporated into a component of the medical device.

Although not to be limited in theory, the decellularized extracellular matrix provides a microenvironment and contains important biological materials that promote the efficient and effective repair, regeneration and/or strengthening of cells, tissues or organs.

For clarity of disclosure, and not by way of limitation, the detailed description of the invention is divided into the subsections which follow.

5.1 Decellularized Extracellular Matrix of Conditioned Body Tissue

5.1.1 Source of Body Tissue

Suitable animal body tissue from which the decellularized extracellular matrix material of the present invention is produced includes body tissues originally from syngeneic, allogeneic or xenogenic sources. The body tissue may be obtained from various animal sources. These animals include, but are not limited to, non-primate (e.g., cows, pigs, horses, chickens, cats, dogs, rats, etc.) and primate (e.g., monkeys and humans). The body tissue may be obtained at approved slaughterhouses from animals fit for human consumption or from herds of domesticated animals maintained for the purpose of providing tissues or organs. Preferably, the body tissue is handled in a sterile manner, and any further dissection of the body tissue is carried out under aseptic conditions. A preferred source of the body tissue is non-human. When the implants are obtained from a human, the donor may be the recipient, the donor may be genetically related to the recipient, or the donor may be unrelated to the recipient. In specific embodiments, the donor is tested for compatibility with the recipient.

Progenitor cells (e.g., endothelial progenitor cells), stem cells (e.g., mesenchymal, hematopoietic, neuronal), stromal cells, parenchymal cells, undifferentiated cells, embryonic cells, fibroblasts, macrophage, and satellite cells are particularly preferred for conditioning using the methods of the present invention. In preferred embodiments, body organs that are useful in the present invention include, but are not limited to, brain, heart, lung, liver, pancreas, stomach, large or small intestine, kidney, bladder, uterus, bone marrow, etc.

The body tissue suitable for the present invention can be grouped into four general categories: (1) epithelial tissue, (2) connective tissue, (3) muscle tissue, and (4) nerve tissue. Epithelial tissue covers or lines all body surfaces inside or outside the body. Examples of epithelial tissue include, but are not limited to, the skin, epithelium, dermis, and the mucosa and serosa that line the body cavity and internal organs, such as the heart, lung, liver, kidney, intestines, bladder, uterine, etc. Connective tissue is the most abundant and widely distributed of all tissues. Examples of connective tissue include, but are not limited to, vascular tissue (e.g., arteries, veins, capillaries), blood (e.g., red blood cells, platelets, white blood cells), lymph, fat, fibers, cartilage, ligaments, tendon, bone, teeth, omentum, submucosa, peritoneum, mesentery, meniscus, conjunctiva, dura mater, umbilical cord, etc. Muscle tissue accounts for nearly one-third of the total body weight and consists of three distinct subtypes: striated (skeletal) muscle, smooth (visceral) muscle, and cardiac muscle. Examples of muscle tissue include, but are not limited to, myocardium (heart muscle), skeletal, intestinal wall, etc. The fourth primary type of tissue is nerve tissue. Nerve tissue is found in the brain, spinal cord, and accompanying nerve. Nerve tissue is composed of specialized cells called neurons (nerve cells) and neuroglial or glial cells.

5.1.2 Conditioning of Body Tissue

The present invention provides methods for conditioning body tissue using one or more biological, chemical, pharmaceutical, physiological and/or mechanical manipulation. Specifically, conditioning is used to make the body tissue either over-express or under-express a biological material of interest as compared to the amount of such biological material that the body tissue would express absent conditioning, or to express a protein or biological material otherwise not present in the tissue. In certain embodiments, the conditioning modifies the production of biological materials that enhance the effectiveness or temporal sequence of repairing, regenerating or strengthening defective, diseased, damaged or ischemic tissues or organs in a subject. In certain other embodiments, the conditioning modifies the production of biological materials that increase the metabolic synthesis of and/or phenotypic expression in endogenous cell populations. The anti-adhesion, bioadhesive, bioresorptive, antithrombogenic, and other physical properties of the body tissue can also be varied as needed by the conditioning process.

Preferably, the conditioning modifies the body tissue's production of extracellular matrix proteins, growth factors, angiogenesis factors, cytokines, morphogens (a biologically active material that is capable of inducing the developmental cascade of cellular and molecular events that culminate in the formation of new, organ-specific tissue), etc., and/or micro-architecture of extracellular matrix components. Examples of the biological material of interest to the present invention include, but are not limited to, vascular endothelial growth factor (VEGF), transforming growth factor (TGF), fibroblast growth factor (FGF), epidermal growth factor (EGF), cartilage growth factor (CGF), nerve growth factor (NGF), keratinocyte growth factor (KGF), skeletal growth factor (SGF), osteoblast-derived growth factor (BDGF), hepatocyte growth factor (HGF), insulin-like growth factor (IGF), cytokine growth factors (CGF), platelet-derived growth factor (PDGF), hypoxia inducible factor-1 (HIF-1), stem cell derived factor (SDF), stem cell factor (SCF), endothelial cell growth supplement (ECGS), granulocyte macrophage colony stimulating factor (GM-CSF), growth differentiation factor (GDF), integrin modulating factor (IMF), calmodulin (CaM), thymidine kinase (TK), tumor necrosis factor (TNF), growth hormone (GH), bone morphogenic protein (BMP) (e.g., BMP-2, BMP-3, BMP-4, BMP-5, BMP-6 (Vgr-1), BMP-7 (PO-1), BMP-8, BMP-9, BMP-10, BMP-11, BMP-12, BMP-14, BMP-15, BMP-16, etc.), matrix metalloproteinase (MMP), tissue inhibitor of matrix metalloproteinase (TIMP), cytokines, interleukin (e.g., IL-1, IL-2, IL-3, IL-4, IL-5, IL-6, IL-7, IL-8, IL-9, IL-10, IL-11, IL-12, IL-15, etc.), lymphokines, interferon, integrin, collagen (all types), elastin, fibrillins, fibronectin, vitronectin, laminin, glycosaminoglycans, proteoglycans, transferrin, cytotactin, cell binding domains (e.g., RGD), tenascin, Serca2, phospholamban repressors, beta-blockers, and ACE inhibitors.

More than one conditioning process may be performed, sequentially or simultaneously. The conditioning of the body tissue can be conducted in vivo, in situ or in vitro. Conditioning the body tissue while it is still in the donor animal has the advantage of retaining the complexity afforded by in vivo remodelling.

Alternatively, after the body tissue is harvested, the biologically active material composition and histoarchitectural property of the body tissue may be modified without in vivo manipulation. When conditioning is performed after the body tissue is isolated or harvested from the donor animal, i.e., in vitro, the body tissue is cultured for a period of time, for example, in a bioreactor. The advantage of in vitro conditioning is that the process is easily monitored and that changes to the biologically active material composition and histoarchitectural property of the body tissue is easily assessed.

Regardless of whether the body tissue is conditioned in vivo or in vitro, or before or after the body tissue is harvested, the conditioned body tissue should be allowed a selected period of time to produce the desired biological material in an amount different than the amount that is produced by an unconditioned body tissue. Preferably, the conditioned body tissue produces at least 5%, at least 10%, at least 25%, at least 50%, at least 100%, at least two times, at least five times, or at least ten times more or less biological material than a body tissue absent conditioning.

In another embodiment, the body tissue is conditioned to express a protein or biological material otherwise not present in the tissue.

In certain preferred embodiments, the conditioned body tissue can be further processed before or after decellularization. In a specific embodiment, a therapeutic agent may be delivered to the body tissue before or after conditioning. Preferably, the therapeutic agent is useful for treating a disease or condition that requires tissue or organ repair, restoration and/or strengthening.

5.1.2.1 Biological Conditioning

The body tissue of a donor animal can be biologically conditioned by genetic engineering to effect a desired change in composition or amount of biologically active material in the body tissue. For instance, the body tissue may be transfected with a nucleic acid that encodes a biological material of interest (see International Publication No. WO 98/28406). The body tissue of a donor animal can also be biologically conditioned using a number of in vitro culture conditions to effect changes to the histoarchitecture of the body tissue and/or composition of biologically active materials in the body tissue. In preferred embodiments, the in vitro biological conditioning includes the use of a bioreactor. In a specific embodiment, the conditioned body tissue is continuously cultured in the bioreactor while toxic metabolic byproducts are removed.

In general, cells in the body tissue of an animal can be transfected in vivo or in vitro with genetic material using any appropriate means such as direct injection of viral vectors, as discussed further in detail below, delivery into the local blood supply (see International Publication Nos. WO 98/58542 and WO 99/55379, each of which is incorporated herein by reference in its entirety), the use of delivery vectors (e.g., liposome) or chemical transfectants, and physico-mechanical methods such as electroporation and direct diffusion of nucleic acid. The transfected body tissue is subsequently cultured for a period of time during which the composition or amount of at least one biological material in the body tissue is changed.

For general reviews of the methods of gene transfer, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217, each of which is incorporated herein by reference in its entirety. Delivery of the nucleic acid into a donor body tissue may be either in vivo, in which case the donor body tissue is exposed to the nucleic acid or nucleic acid-carrying vector or delivery complex before being harvested from the donor animal; or in vitro, in which case, the donor body tissue may first be harvested from the donor animal and then transformed with the nucleic acid in vitro. These two approaches are known, respectively, as in vivo or in vitro gene transfer.

In one embodiment, the nucleic acid is directly administered in vivo, where it is expressed to produce a biologically active material. This can be accomplished by any of numerous methods known in the art, e.g., by constructing it as part of an appropriate nucleic acid expression vector and administering it so that it becomes intracellular, by infection using a defective or attenuated retroviral or other viral vector (see infra. and U.S. Pat. No. 4,980,286), by direct injection of naked DNA, by use of microparticle bombardment (e.g., a gene gun; Biolistic, Dupont), by encapsulation in biopolymers (poly-β-1-4-N-acetylglucosamine polysaccharide; see U.S. Pat. No. 5,635,493), by administering it in linkage to a peptide or ligand which is known to enter the nucleus, by receptor-mediated endocytosis (see, e.g., Wu and Wu, 1987, J. Biol. Chem. 262:4429-4432), or by coating with lipids.

Viral vectors include adenoviruses, gutted adenoviruses, adeno-associated virus, retroviruses, alpha virus (Semliki Forest, Sindbis, etc.), lentiviruses, herpes simplex virus, replication competent viruses (e.g., ONYX-015), and hybrid vectors. Non-viral vectors include artificial chromosomes and mini-chromosomes, plasmid DNA vectors (e.g., pCOR), cationic polymers (e.g., polyethyleneimine, polyethyleneimine (PEI)) graft copolymers (e.g., polyether-PEI and polyethylene oxide-PEI), neutral polymers PVP, SP1017 (SUPRATEK), lipids or lipoplexes, nanoparticles and microparticles with and without targeting sequences such as the protein transduction domain (PTD).

Adenoviruses, in particular, are especially attractive vehicles for delivering genes to respiratory epithelia where they cause a mild disease. Other targets for adenovirus-based delivery systems are liver, the central nervous system, endothelial cells, and muscle. The use of adenoviruses has the advantage of being capable of infecting non-dividing cells. Kozarsky and Wilson present a review of adenovirus-based gene transfer (1993, Current Opinion in Genetics and Development 3:499-503). Bout et al. demonstrate the use of adenovirus vectors to transfer genes to the respiratory epithelia of rhesus monkeys (1994, Human Gene Therapy 5:3-10). Other instances of the use of adenoviruses in gene transfer can be found in Rosenfeld et al., 1991, Science 252:431-434; Rosenfeld et al., 1992, Cell 68:143-155; and Mastrangeli et al., 1993, J. Clin. Invest. 91:225-234. Adeno-associated virus (AAV) has also been proposed for use in gene transfer (see Walsh et al., 1993, Proc. Soc. Exp. Biol. Med. 204:289-300).

Genetically ex vivo modified cells (e.g., stem cells, fibroblasts, myoblasts, satellite cells, pericytes, cardiomyocytes, skeletal myocytes, macrophage) may be delivered to the tissue. The cells then condition the matrix.

Another way to transport the gene that encodes the biologically active material into the body tissue involves chemical or physical treatment of the cells in the body tissue to increase the potential for gene uptake and allowing the gene to be directly introduced into the nucleus or target the gene to a cell receptor. In certain embodiments, these include the use of vectors that exploit receptors on the surface of cells using liposomes, lipids, ligands for specific surface receptors, cell receptors, calcium phosphate and other chemical mediators, microinjections, electroporation, sperms, and homologous recombination. Liposomes are commercially available from Gibco BRL, for example, as LIPOFECTION® and LIPOFECTACE®, which are formed of cationic lipids such as N-[1-(2,3 dioleyloxy)-propyl]-nmnm-trimethylammonium chloride (DOTMA) and dimethyl dioctadecylammonium bromide (DDAB). Numerous methods for making liposomes are also known to those skilled in the art.

In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted for cell specific uptake and expression, by targeting a specific receptor (see, e.g., International Publications Nos. WO 92/06180, WO 92/22635, WO92/20316, and WO93/14188, each of which is incorporated herein by reference in its entirety). Alternatively, the nucleic acid can be introduced intracellularly and incorporated within host cell DNA for expression, by homologous recombination (Koller and Smithies, 1989, Proc. Natl. Acad. Sci. USA 86:8932-8935; Zijlstra et al., 1989, Nature 342:435-438).

The invention also relates to a method for biologically conditioning body tissue by inoculating the body tissue with a solution having microorganisms, where the microorganisms are selected to produce chemicals that process the tissue. The body tissue is incubated with the inoculated microorganisms under conditions that are effective for processing the body tissue by the chemicals produced by the microorganisms. The body tissue may be subsequently treated to substantially remove or inactivate the microorganisms (see U.S. Pat. No. 6,121,041).

In other embodiments, the tissue or organ may be transformed with one or more different recombinant nucleic acid molecules, so that the cells within the tissue or organ may express at least one recombinant protein. In another embodiment, a single cell in the tissue or organ may be transfected with a single recombinant nucleic acid molecule that expresses at least one protein, which can be under the control of the same transcription control sequences or under the control of different transcription control sequences. Methods commonly known in the art of recombinant DNA technology which may be used are described in Ausubel et al. (eds.), 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al. (eds), 1994, Current Protocols in Human Genetics, John Wiley & Sons, NY.

In preferred embodiments, the invention creates in the tissue or organ localized depots for a biologically active material. The tissue or organ serves to concentrate the binding of biologically active material such as drugs that are introduced, for example, locally or systemically. This is accomplished by upregulating the production of anionic/cationic species, specific antibody recognition sequences, cell receptors, etc., in the tissue or organ. For example, the conditioned body tissue which comprises cells with a highly positively charged matrix would enhance the localization of nucleic acid at this site. This would sustain nucleic acid delivery, improve transfection and reduce degradation of the nucleic acid. In a specific embodiment, the depot would provide localization for biologically active material for the treatment of ischemia. In another specific embodiment, the depot provide localization for biologically active material listed supra and α-adrenergic blockers, β-adrenergic blockers, α-adrenergic agonists, α-1 adrenergic antagonists, AMP kinase activators, angiotensin converting enzyme (ACE) inhibitors, angiotensin II receptor antagonists, antiarrhythmic agents, anticoagulation agents, antiplatelet aggregation agents, antidiabetic agents, antioxidants, anti-inflammatory agents, beta blockers, bile acid sequestrants, calcium channel blockers, calcium antagonists, CETP inhibitors, cholesterol reducing agents/lipid regulators, drugs that block arachidonic acid conversion, duretics, estrogen replacement agents, inotrophic agents, fatty acid analogs, fatty acid synthesis inhibitors, fibrates, histidine, nicotine acid derivatives, nitrates, peroxisome proliferator activator receptor agonists or antagonists, ranolzine, statins, thalidomide, thiazolidinediones, thrombolytic agents, vasodilators, and vassopressors.

The form and amount of nucleic acid envisioned for use depends on the type of biologically active material and the desired effect and can be readily determined by one skilled in the art. For transfection of cells without or minimized toxic effects see U.S. Pat. No. 6,284,880.

Nucleic acids that are useful as biologically active materials for gene transfer in the present invention include, e.g., DNA and RNA sequences, that have a therapeutic or prophylactic effect after being taken up by the cells of a tissue or an organ. In one embodiment, the nucleic acid comprises an expression vector that expresses a biologically active material. In another embodiment, the nucleic acid comprises a part of an expression vector that expresses a protein or a functionally active fragment, derivative or analog thereof, or a chimeric protein (see International Publication No. WO 01/90158). The nucleic acids may encode RNA interference sequences that shut down mRNA production.

In specific embodiments, the nucleic acid encodes a sequence without a leader sequence which produces an intracellular protein. In other specific embodiments, the nucleic acid encodes a sequence with a leader sequence which produces an intercellular protein. In a specific embodiment, the nucleic acid encodes a biologically active material or a functionally active fragment, derivative or analog thereof.

Preferably, the nucleic acid useful in the invention encodes for polypeptides. A polypeptide is understood to be any translation product of a polynucleotide regardless of size, and whether modified or not. The polypeptide may be modified by, e.g., glycosylation, acetylation, formylation, pegylation, phosphorylation, amidation, derivatization by known protecting/blocking groups, proteolytic cleavage, linkage to a cellular ligand or other protein, etc. In specific embodiments, one or more amino acid residues in the amino acid sequence of the polypeptide, preferably non-conserved amino acid residues, may include insertion, deletion and/or substitution with a different amino acid residue. These polypeptides may include, for example, those polypeptides that are biologically active in the body tissue of the donor and/or recipient animal.

The polypeptides, proteins, or functionally active fragments, derivatives, and analogs thereof, that are encoded by nucleic acids used in gene transfer include without limitation, structural proteins, growth factors and cytokines which promotes or enhances repair, regeneration or strengthening of defective, diseased, damaged or ischemic cells, tissues or organs.

Most preferably, genes that are useful for the present invention encode proteins such as vascular endothelial growth factor (VEGF), transforming growth factor (TGF), fibroblast growth factor (FGF), epidermal growth factor (EGF), cartilage growth factor (CGF), nerve growth factor (NGF), keratinocyte growth factor (KGF), skeletal growth factor (SGF), osteoblast-derived growth factor (BDGF), hepatocyte growth factor (HGF), insulin-like growth factor (IGF), cytokine growth factors (CGF), platelet-derived growth factor (PDGF), hypoxia inducible factor-1 (HIF-1), stem cell derived factor (SDF), stem cell factor (SCF), endothelial cell growth supplement (ECGS), granulocyte macrophage colony stimulating factor (GM-CSF), growth differentiation factor (GDF), integrin modulating factor (IMF), calmodulin (CaM), thymidine kinase (TK), tumor necrosis factor (TNF), growth hormone (GH), bone morphogenic proteins (BMP), matrix metalloproteinase (MMP), tissue inhibitor of matrix metalloproteinase (TIMP), cytokines, interleukins, lymphokines, interferon, integrin, collagen (all types), elastin, fibrillins, fibronectin, heparin, laminin, glycosaminoglycans, vitronectin, proteoglycans, transferrin, cytotactin, cell binding domains (e.g., RGD), and tenascin. Other genes that are useful in the present invention include those that promote angiogenesis, modulate inflammation, and increase cell adhesion, proliferation and regeneration.

In a particularly preferred embodiment, genes encoding for elastin may be used to increase elastic properties of the tissue being implanted. The amount of elastin would be tailored to ultimately result in a suitable material for stent coatings, i.e., to produce an elongation property necessary to comply with stent expansion.

Antisense and ribozyme molecules which inhibit expression of a target gene can also be used in accordance with the invention. For example, in a preferred embodiment, antisense RNA molecules which inhibit the expression of major histocompatibility gene complexes (HLA) have been shown to be most versatile with respect to modulating immune responses. Furthermore, appropriate ribozyme molecules can be designed as described, e.g., Hascloff et al., 1988, Nature 334:585-591; Zaug et al., 1984, Science 224:674-578; and Zaug and Cech, 1986, Science 231:470-475. Still further, triple helix molecules can be utilized in reducing the level of target gene activity. These techniques are described in detail by L. G. Davis et al., eds, Basic Methods in Molecular Biology, 2^{nd} ed., Appleton & Lange, Norwalk, Conn. 1994. Using any of the foregoing techniques, the expression of MRC class II molecules can be knocked out in order to reduce the risk of rejection of the tissue constructs described herein.

5.1.2.2 Chemical Conditioning

The body tissues may be chemically conditioned to effect a desired change in the composition of biologically active material and/or the histoarchitechture of the body tissue. In one embodiment, the body tissue may be chemically conditioned by incubating the body tissue in vitro with an isosmotic, hypotonic and/or hypertonic solution (see, e.g., U.S. Pat. No. 5,855,620 and International Publication No. WO 96/32905). Studies have shown that changes in cellular osmolality appear to directly influence cell metabolism such as lipolysis (Bilz et al., 1999, Metabolism 48(4):472-6) or protein synthesis (Schmid, 1986, Klin Wochenschr 64(1):23-8; Yates et al., 1982, J. Biol. Chem. 257(24):15030-4).

In other embodiments, the body tissue may be detoxified with reducing agents including, for example, inorganic sulfur-oxygen ions, such as bisulfate and thiosulfate, organic sulfates, amines, ammonia/ammonium, and surfactants. Chemical solutions may also be added to modulate the salinitiy, pH (acidity and alkalinity), ion concentration (e.g., potassium, calcium, magnesium, phosphorous, sodium, nitrate, etc.), blood variables, plasma volume, and oxygen level of the body tissue to facilitate a change in the composition or amount of biologically active materials. Preferably, the body tissue is chemically conditioned to promote protein synthesis, cell proliferation, tissue regeneration and strengthening or make the cells more susceptible to biological, physiological and/or mechanical conditioning.

b 5.1.2.3 Pharmaceutical Conditioning

Another aspect of the invention relates to the pharmaceutical conditioning of the body tissue by delivering a therapeutic agent to the body tissue. In one embodiment, the therapeutic agent is delivered to the body tissue before the body tissue is harvested. In another embodiment, the therapeutic agent is delivered to the body tissue after the body tissue is harvested.

Therapeutic agents include those that are effective at treating, managing or preventing a disease or condition that requires tissue or organ repair, restoration and/or strengthening. Other therapeutic agents include those that that promote angiogenesis, modulate inflammation, and increase cell adhesion, proliferation and regeneration. Examples of therapeutic agents include, but are not limited to, vascular endothelial growth factor (VEGF), transforming growth factor (TGF), fibroblast growth factor (FGF), epidermal growth factor (EGF), cartilage growth factor (CGF), nerve growth factor (NGF), keratinocyte growth factor (KGF), skeletal growth factor (SGF), osteoblast-derived growth factor (BDGF), hepatocyte growth factor (HGF), insulin-like growth factor (IGF), cytokine growth factors (CGF), platelet-derived growth factor (PDGF), hypoxia inducible factor-1 (HIF-1), stem cell derived factor (SDF), stem cell factor (SCF), endothelial cell growth supplement (ECGS), granulocyte macrophage colony stimulating factor (GM-CSF), growth differentiation factor (GDF), integrin modulating factor (IMF), calmodulin (CaM), thymidine kinase (TK), tumor necrosis factor (TNF), growth hormone (GH), bone morphogenic proteins (BMP), matrix metalloproteinase (MMP), tissue inhibitor of matrix. metalloproteinase (TIMP), cytokines, interleukins, lymphokines, interferon, integrin, collagen (all types), elastin, fibrillins, fibronectin, laminin, glycosaminoglycans, vitronectin, proteoglycans, transferrin, cytotactin, cell binding domains (e.g., RGD), tenascin, anti-inflammatory drugs, α-adrenergic blockers, β-adrenergic blockers, α-adrenergic agonists, α-1 adrenergic antagonists, AMP kinase activators, angiotensin converting enzyme (ACE) inhibitors, angiotensin II receptor antagonists, antiarrhythmic agents, anticoagulation agents, antiplatelet aggregation agents, antidiabetic agents, antioxidants, anti-inflammatory agents, beta blockers, bile acid sequestrants, calcium channel blockers, calcium antagonists, CETP inhibitors, cholesterol reducing agents/lipid regulators, drugs that block arachidonic acid conversion, duretics, estrogen replacement agents, inotrophic agents, fatty acid analogs, fatty acid synthesis inhibitors, fibrates, histidine, nicotine acid derivatives, nitrates, peroxisome proliferator activator receptor agonists or antagonists, ranolzine, statins, thalidomide, thiazolidinediones, thrombolytic agents, vasodilators, vassopressors, vitamins, antioxidants, herbal extracts, metals, etc.

The body tissue may be conditioned pharmaceutically either when the donor subject is undergoing or has already undergone a medication or treatment, wherein as a result of the medication or treatment, the production of biological materials in the body tissue is effected.

5.1.2.4 Physiological Conditioning

The body tissue may be physiologically conditioned to effect a process or function of the body tissue. In particular, the body tissue may be physiologically conditioned to increase or decrease the level and/or rate of production of a biologically active material in the body tissue by subjecting the body tissue to temperature changes that affect chemical and protein synthesis in the cells (see e.g., Tibbett et al., 2002, Mycorrhiza 12(5):249-55).

In one embodiment, the body tissue is physiologically conditioned by cryopreservation and subsequent thawing of the body tissue as described in U.S. Pat. No. 6,291,240, which is incorporated by reference herein in its entirety. Specifically, cryopreservation and subsequent thawing (“cryopreservation/thaw cycle”) induced the cells of the body tissue to produce useful regulatory proteins, such as, growth factors, cytokines, and stress proteins (e.g., GRP78 and HSP90). Stress proteins are known to stabilize cellular structures and render the cells resistant to adverse conditions.

In a specific embodiment, the tissue and organs may be cryopreserved or frozen to below −50° C. to −180° C., preferably, to below −50° C., more preferably, to below −65° C. to −70° C. In another specific embodiment, the body tissue may be cryopreserved by adding glycosaminoglycan or other extracellular matrix proteins and using freezing schedule designed to maximize retention of tissue cell viability and biomechanical properties during and after the freezing process, and following a thawing schedule which maximizes cell viability. The cryopreserving agent comprises a cell-penetrating organic solute, which is preferably dimethylsulfoxide, and a glycosaminoglycan, which is preferably chondroitin sulphate, in an amount sufficient to cryopreserve the musculoskeletal tissue such as ligaments, tendons and cartilage (see International Publication No. WO 91/06213).

In yet another embodiment, the body tissue is subjected to physiological stresses such as oxygen deprivation or nutrient deficiency. The stress imposed on the tissue or organ by the oxygen or nutrient deprivation induces the production of regulatory proteins in the tissue or organ and in turn changes the compositions of the biologically active material and physical structure of the body implant.

Alternatively, U.S. Pat. No. 5,824,080 describes the use of photodynamic therapy (PDT), a technique to produce cytotoxic free radicals, to condition arterial tissues. The collagens in the matrix may be cross-linked using photooxidative catalysis and visible light and therefore, add mechanical strength and/or resilience to the body tissue.

5.1.2.5 Mechanical Conditioning

Tissues respond to mechanical forces by remodelling the extracellular matrix. The magnitude and direction of mechanical force will determine the extent and type of remodelling. For example, increased stress on bones results in an increase in bone mass. Accordingly, artificial stressing of a tissue or organ that is to be harvested for the present invention modifies the properties and compositions of biologically active materials of the tissue or organ. The mechanical force can be repeatedly applied over a period of time until the desired amount of biological active material is obtained.

In a specific embodiment, a portion of the small intestine of a donor animal, preferably a pig, may be mechanically conditioned by placing a balloon inside the portion of the small intestine. The balloon is inflated such that it stretches the intestinal wall. Preferably, the inflation or deflation of the balloon may occur in a cyclic fashion. More preferably, the inflation only occurs during certain periods of time during the day, thus allowing the animal's digestive system to function normally when the balloon is deflated.

Other methods of mechanically conditioning the body tissue includes the use of standard clips to create tension in the body tissue. In another specific embodiment, the body tissue is mechanically conditioned by the application of strain, wherein cell division is facilitated and the activity of matrix metalloproteinases (MMPs) are improved (see International Publication No. WO 02/62971). In yet another specific embodiment, the body tissue is subject to a hydrostatic and/or hydrodynamic force as described in U.S. Pat. No. 6,197,296, which is incorporated herein by reference in its entirety.

In yet another specific embodiment, the body tissue is subject to electroprocessing techniques, including electrospin, electrospray, electroaerosol, and electrosputter (see International Publication Nos. WO 02/40242 and WO 02/18441). Centrifugation, electrical stimulation, electromagnetic forces (e.g., seeding tissue and/or cells with magnetic particles), hydrostatic or hydrodynamic forces, sound waves, and ultrasound waves may also be used to manipulate the amount or composition of biologically active materials in the body tissue. In a specific embodiment, the electrical stimulation is generated with conductive wires connected to an electric potential which cause changes by varying the electric field or by causing mechanical forces (e.g., muscle contraction). In another specific embodiment, the electromagnetic forces and/or strains are generated by applying an electromagnetic field. In another specific embodiment, the hydrostatic or hydrodynamic forces are generated by first inserting a catheter or cannula into the tissue or organ; then forcing saline or another biologically inert fluid into the tissue and subsequently removing the same from the tissue such that the forces from the pressurized fluid conditions the tissue. In yet another specific embodiment, the sound wave and ultrasound waves are produced by commercially available spealers or transducers.

This invention also provides an in vitro method for mechanically conditioning tissue in an oriented manner (see U.S. Pat. Nos. 5,765,350, 5,700,688 and 5,521,087). For example, connective body tissues may be aligned along a defined axis to produce an oriented tissue-equivalent having increased mechanical strength in the direction of the axis. The tensile strength of collagen in a body tissue can also be improved by cross-linking or plasticizing collagen thread or thread construct with a plasticizing agent, imparting a tensile load to the collagen thread or construct to strain the collagen thread, and then allowing the strain in the thread to decrease by stress-relaxation or by creep (see U.S. Pat. No. 5,718,012 and International Publication No. WO 97/45071). The amount of biological material may be measured before, during and/or after the conditioning.

Biological, chemical, or pharmaceutical conditioning may be enhanced by use of ultrasound or iontophoresis during delivery to the tissue to be conditioned.

5.1.3 Culturing the Conditioned Body Tissue

The conditioned body tissue may be cultured over a period of time to allow changes in the biochemical composition and histoarchitecture to occur. Preferably, the conditioned body tissue is allowed a period of time to produce a biological material in an amount that is different than the amount that would be produced by an unconditioned body tissue.

The period of time in culture varies depending on the type of conditioning and also the extent of change desired. In specific embodiments, the conditioned body tissue may be cultured for at least 10 minutes, at least 30 minutes, at least 1 hour, at least 2 hours, at least 3 hours, at least 4 hours, at least 6 hours, at least 8 hours, at least 12 hours, at least 24 hours, at least 2 days, at least 4 days, at least 6 days, at least 8 days, at least 10 days, at least 1 week, at least 2 weeks, at least 3 weeks, at least 1 month, at least 2 months, or at least 3 months.

For conditioning process that are carried out in vitro, the body tissue may be grown in multicavity bag or bioreactors which provide low shear to the tissue. The bioreactor designs useful for the present invention are disclosed in, e.g., U.S. Pat. Nos. 4,988,623; 5,026,650; 5,153,131; and 5,928,945. In a preferred embodiment, a horizontal rotating wall vessel (RWV) bioreactor is used. The RWV bioreactor is described in U.S. Pat. No. 5,026,650 and is incorporated by reference herein. In preferred embodiments, culture medium such as supplemented Dulbeccos modified Eagle medium (DMEM) (e.g., Life Technologies, Grand Island, N.Y.) may be used.

5.1.4 Assays for Monitoring the Effects of Conditioning

Changes in the amount of biologically active material subsequent to various conditioning methods may be assayed using methods known in the art. For example, mRNA levels for any factors may be determined using standard techniques in the art such as the quantitative reverse transcript TaqMan® polymerase chain reaction (QRTPCR) (see, e.g., Holland et al., 1991, Proc. Natl. Acad. Sci. USA 88:7276-7289 and Lee et al., 1993, Nucl. Acids Res. 21:3761-3766). Protein levels can also be determined by techniques such as Western blots, standard ELISA assays, and biological activity assays such as the chick chorioallantoic membrane (CAM) assay.

5.1.5 Decellularized Extracellular Matrix

Decellularization generally refers to the removal of all cells, cellular components, and other non-extracellular matrix components (e.g., serum, fat) while leaving intact an extracellular matrix (ECM) component. It is believed that the process of decellularization can reduce or eliminate immune response associated with the cells as well as the cellular components. Acellular vascular tissues have been suggested to be ideal natural biomaterials for tissue repair and engineering (Schmidt and Baier, 2000, Biomaterials 21:2215-31).

Several means of reducing the viability of native cells in tissues and organs are known, including physical, chemical, and biological methods (see, e.g. Kaushal et al., 2001, Nature Medicine 7(9):1035; Schmidt et al., supra; and U.S. Pat. No. 5,192,312, which are incorporated herein by reference). Such methods may be employed in accordance with the process described herein. However, in preferred embodiments, the decellularization technique employed should not result in gross disruption of the anatomy of the body tissue or substantially alter its biomechanical properties or histoarchitecture. Similarly, the treatment of the body tissue to produce a decellularized extracellular matrix should also not leave a cytotoxic environment that inhibits subsequent repopulation of the extracellular matrix with cells from a recipient after implantation of the decellularized extracellular matrix. Decellularization by physical, chemical and/or biological treatments are optimized to preserve as much as possible the biological material of interest and more importantly, the microstructure of the extracellular matrix.

Extracellular matrix may be isolated from the conditioned body tissue using a physical technique, including but not limited to centrifugation, rinsing, agitation, freeze-thaw, sedimentation, dialysis, electrical stimulation, electromagnetic forces, hydrostatic or hydrodynamic forces, blasting with sound waves, and ultrasonication. For example, the conditioned body tissue may be minced to disrupt the cell membrane and disorganize cellular components. The minced body tissue may then be centrifuged with a liquid preparation, preferably Histopaque and more preferably water or saline, which separates components of different densities. In preferred embodiments, the speed for centrifugation ranges from 100 to 10,000 g, and more preferably, from 2,500 to 7,500 g, for between 5 to 20 minutes. The components in the resulting suspension may then be separated using filters of specific pore size. In one embodiment, the filter is of a pore size, preferably of 70 to 250 μm, that allows the extracellular matrix to pass through. In another embodiment, the filter is of a pore size, preferably of 20 to 100 μm, that retains the extracellular matrix and larger components. Filtration is carried out in one step or a series of steps.

It has been reported that modification of the magnitude of the membrane dipole potential using compounds such as cholesterol, phloretin, and 6-ketocholestanol may also influence binding capacity and disrupts membrane domains. (Asawakarn T. et al., 2001, J. Biol. Chem. 276:38457-63). Accordingly, the present invention further relates to methods for decellularizing conditioned body tissue by agitating cellular membrane potential using electrical (e.g., voltage) means.

In another specific embodiment, the formation of intracellular ice is used to decellularize the conditioned body tissue. For example, vapor phase freezing (slow rate of temperature decline) of the body tissue reduces the cellularity of the body tissue as compared to liquid phase freezing (rapid). However, slow freezing processes, in the absence of cryoprotectant, may result in tissue disruption such as cracking. Colloid-forming materials may be added during freeze-thaw cycles to alter ice formation patterns in the body tissue. Polyvinylpyrrolidone (10% w/v) and dialyzed hydroxyethyl starch (10% w/v) may be added to standard cryopreservation solutions (DMEM, 10% DMSO, 10% fetal bovine serum) to reduce extracellular ice formation while permitting formation of intracellular ice. This allows a measure of decellularization while affording the collagenase tissue matrix some protection from ice damage.

Alternatively, the conditioned body tissue may be decellularized using a chemical technique. In one embodiment, the conditioned body tissue is treated with a solution effective to lyse native cells. Preferably, the solution may be an aqueous hypotonic or low ionic strength solution formulated to effectively lyse the native tissue cells. Such an aqueous hypotonic solution may be de-ionized water or an aqueous hypotonic buffer. Preferably, the aqueous hypotonic buffer may contain additives that provide suboptimal conditions for the activity of selected proteases, e.g., collagenase, which may be released as a result of cellular lysis. Additives such as metal ion chelators, e.g., 1,10-phenanthroline and ethylenediaminetetraacetic acid (EDTA), create an environment unfavorable to many proteolytic enzymes.

In another embodiment, the conditioned body tissue is treated with a hypotonic lysis solution with protease inhibitors. General inhibitor solutions manufactured by Sigma and Genotech are preferred. Specifically, 4-(2-aminoethyl)-benzene-sulfonyl fluoride, E-64, bestatin, leopeptin, aprotin, PMSF, Na EDTA, TIMPs, pepstatin A, phosphoramidon, and 1,10-phenanthroline are non-limiting examples of preferred protease inhibitors. The hypotonic lysis solution may have include a buffered solution of water, pH 5.5 to 8, preferably pH 7 to 8. In preferred embodiments, the hypotonic lysis solution is free from calcium and zinc ions. Additionally, control of the temperature and time parameters during the treatment of the body tissue with the hypotonic lysis solution, may also be employed to limit the activity of proteases.

In certain embodiments, the body tissue is treated with a detergent. In one embodiment, the body tissue is treated with an anionic detergent, preferably sodium dodecyl sulfate in buffer. In another embodiment, the body tissue is treated with a non-ionic detergent, such as Triton X-100 or 1% octyl phenoxyl polyethoxyethanol, to solubilize cell membranes and fat. In a preferred embodiment, the body tissue is treated with a combination of different classes of detergents, for example, a nonionic detergent, Triton X-100, and an anionic detergent, sodium dodecyl sulfate, to disrupt cell membranes and aid in the removal of cellular debris from tissue.

Steps should be taken to eliminate any residual detergent levels in the extracellular matrix, so as to avoid interference with the latter's ability to repair, regenerate or strengthen defective, diseased, damaged or ischemic tissues or organs. Selection of detergent type and concentration will be based partly on its preservation of the structure, composition, and biological activity of the extracellular matrix.

In other embodiments, extracellular matrix may be isolated from the conditioned body tissue using a biological technique. Various enzymes may be used to eliminate viable native cells from the body tissue. Preferably, the enzyme treatment limits the generation of new immunological sites. For instance, extended exposure of the body tissue to proteases such as trypsin result in cell death. However, because at least a portion of the type I collagen molecule is sensitive to a variety of proteases, including trypsin, this may not be the approach of choice for collagenous grafts intended for implant in high mechanical stress locations.

In one embodiment, the body tissue is treated with nucleases to remove DNA and RNA. Nucleases are effective to inhibit cellular metabolism, protein production, and cell division without degrading the underlying collagen matrix. Nucleases that can be used for digestion of native cell DNA and RNA include both exonucleases and endonucleases. A wide variety of which are suitable for use in this step of the process and are commercially available. For example, exonucleases that effectively inhibit cellular activity include DNase I and RNase A (SIGMA Chemical Company, St. Louis, Mo.) and endonucleases that effectively inhibit cellular activity include EcoR I (SIGMA Chemical Company, St. Louis, Mo.) and Hind III (SIGMA Chemical Company, St. Louis, Mo.). It is preferable that the selected nucleases are applied in a physiological buffer solution which contains ions, such as magnesium and calcium salts, which are optimal for the activity of the nuclease. It is also preferred that the ionic concentration of the buffered solution, the treatment temperature, and the length of treatment are selected to assure the desired level of effective nuclease activity. The buffer is preferably hypotonic to promote access of the nucleases to the cell interiors.

Other enzymatic digestion may be suitable for use herein, for example, enzymes that disrupt the function of native cells in a transplant tissue may be used. For example, phospholipase, particularly phospholipases A or C, in a buffered solution, may be used to inhibit cellular function by disrupting cellular membranes of endogenous cells. Preferably, the enzyme employed should not have a detrimental effect on the extracellular matrix protein. The enzymes suitable for use may also be selected with respect to inhibition of cellular integrity, and also include enzymes which may interfere with cellular protein production. The pH of the vehicle, as well as the composition of the vehicle, will also be adjusted with respect to the pH activity profile of the enzyme chosen for use. Moreover, the temperature applied during application of the enzyme to the tissue should be adjusted in order to optimize enzymatic activity.

In another embodiment, the body tissue is treated so the cells are removed using immunomagnetic bead separation techniques directed to cell surface markers (e.g., integrins, lineage markers, stem cell markers). Immunomagnetic separation (IMS) technology can isolate strains possessing specific and characteristic surface antigens (Olsvik O. et al., 1994, Clin. Microbiol Rev. 7:43-54). Commercially available immunomagnetic separation processes such as Cell Release™ (Sigris Research, Brea, Calif.) was developed to address the need for a fast, general-purpose way to detach intact cells from beads after immunomagnetic separation.

Subsequent to decellularization protocols, the resultant extracellular matrix is washed at least once with suitable chemical solutions, such as saline, protease, enzymes, detergents, alcohols, acidic or basic solutions, salt solutions, etc., to assure removal of cell debris which may include cellular protein, cellular lipids, and cellular nucleic acid, as well as any extracellular debris such as lipids and proteoglycans. Removal of the cellular and extracellular debris reduces the likelihood of the extracellular matrix eliciting an adverse immune response from the recipient upon injection or implantation. For example, the tissue may be incubated in a balanced salt solution such as Hanks' Balanced Salt Solution (HBSS), preferably sterile. The washing process may include incubation at a temperature of between about 2° C. and 42° C., with 4° C. to 25° C. most preferable. The transplant tissue matrix may be incubated in the balanced salt wash solution for up to 10 to 12 days, with changes in wash solution every second or third day. The composition of the balanced salt solution wash, and the conditions under which it is applied to the transplant tissue matrix may be selected to diminish or eliminate the activity of the nuclease or other enzyme utilized during the decellularization process.

Optionally, an antibacterial, an antifungal or a sterilant or a combination thereof, may be included in the balanced salt wash solution to protect the transplant tissue matrix from contamination with environmental pathogens. In certain embodiments, the ECM is sterilized by irradiation, ultraviolet light exposure, ethanol incubation (70-100%), treatment with glutaraldehyde, peracetic acid (0.1-1% in 4% ethanol), chloroform (0.5%), or antimycotic and antibacterial substances.

The extracellular matrix prepared in accordance with the above is free of its native cells, and additionally, cellular and extra-cellular antigen components have been washed out of the extracellular matrix. Preferably, the extracellular matrix has been treated in a manner which limits the generation of new immunological sites in the collagen matrix. In one embodiment, the ECM is obtained as a slurry of small particles. This slurry may eventually be processed into an implant. In another embodiment, the ECM is obtained as an entire or partial structure, such as a sheet, or a tubular member, such as a small intestine.

In addition, the decellularized extracellular matrix may contain a significant portion of the original tissue mass retaining physical properties in regard to strength and elasticity and has components which are largely collagens but also comprise glycosaminoglycans and proteins closely associated with collagen such as the basement membrane complex, laminin, fibronectin, growth factors, and cytokines.

One aspect of the invention further provides the preservation of the decellularized extracellular matrix for later use. The decellularized extracellular matrix can be freeze-dried for prolonged storage. Likewise, the decellularized extracellular matrix can be air-dried by any known standard techniques. In one embodiment, the decellularized extracellular matrix can be concentrated or dehydrated and later reconstituted or rehydrated, respectively, before use. In yet another embodiment, the decellularized extracellular matrix can be used to screen pathogens such as bacteria, virus, and fungus, etc.

In yet another embodiment, the decellularized extracellular matrix is lyophilized. The lyophilized ECM may be in the form of an implant which has pores. Characteristics of the pore structure can be controlled by process parameters. In yet another embodiment, the decellularized extracellular matrix is formed as a gel. Preferably, the proteins are temporarily and reversibly denatured. In yet another embodiment, the decellularized extracellular matrix is precipitated or co-precipitated with other proteins or biologics.

In certain embodiments, the decellularized extracellular matrix is cryopreserved. General techniques for cryopreservation of cells are well-known in the art (see, e.g., Doyle et al., (eds), 1995, Cell & Tissue Culture: Laboratory Procedures, John Wiley & Sons, Chichester; and Ho and Wang (eds), 1991, Animal Cell Bioreactors, Butterworth-Heinemann, Boston, each of which is incorporated herein by reference). Preferably, the tissue or organ is thawed rapidly before use, in a water bath at 34° C. to 37° C., to avoid damage to the cells. Cryopreservation of decellularized extracellular matrix would assure a supply or inventory of substantially non-immunogenic extracellular matrices which, upon thawing, would be ready for further treatment according to the subsequent steps of this invention, or further processed as desired to provide an implant tissue product. For example, extracellular matrices may be inventoried until such time as the particular cells to be employed during repopulation are identified. This may be of particular utility when the extracellular matrix is to be repopulated with cells derived from the recipient or other cells selected for use based on their immunological compatibility with a specific recipient. The ECM may also be used in combination with cells.

5.2 Uses of the Decellularized Extracellular Matrix

The present invention further provides methods for repairing, regenerating or strengthening cells, tissues or organs. In particular, the invention relates to methods for formulating the decellularized extracellular matrix as pharmaceutical compositions, body implants, tissue regeneration scaffolds, and medical devices. Although decellularized extracellular matrix of conditioned body tissue is described in detail, it is not necessary that the decellularized extracellular matrix be of conditioned body tissue. For example, the decellularized extracellular matrix may be of body tissue that has not be conditioned as described herein. Accordingly, although some of the uses of the decellularized extracellular matrix may be described as a use for a decellularized extracellular matrix of a conditioned body tissue, decellularized extracellular matrix of a non-conditioned body tissue may alternatively be used.

In certain embodiments, the decellularized extracellular matrix of body tissue, including conditioned body tissue, may be used to treat defective, diseased, damaged or ischemic tissues or organs which include, but are not limited to, head, neck, eye, mouth, throat, esophagus, chest, bone, ligament, cartilage, tendons, lung, colon, rectum, stomach, prostate, breast, ovaries, fallopian tubes, uterus, cervix, testicles or other reproductive organs, hair follicles, skin, diaphragm, thyroid, blood, muscles, bone marrow, heart, lymph nodes, blood vessels, large intestine, small intestine, kidney, liver, pancreas, brain, spinal cord, and the central nervous system.

In particular, the decellularized extracellular matrix of body tissue, including conditioned body tissue, of the present invention may be used to treat diseases that may benefit from improved angiogenesis, cell proliferation and tissue regeneration. Such diseases or conditions include, but are not limited to, burns, ulcer, trauma, wound, bone fracture, diabetes, psoriasis, arthritis, asthma, cystitis, inflammation, infection, ischemia, restenosis, stricture, atherosclerosis, occlusion, stroke, infarct, aneurysm, abdominal aortic aneurysm, uterine fibroid, urinary incontinence, vascular disorders, hemophilia, cancer, and organ failure (e.g., heart, kidney, lung, liver, intestine, etc.).

In a specific embodiment, the present invention regenerates or replaces at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, at least 10%, at least 5%, or at least 1% of defective, diseased, damaged or ischemic cells from the affected tissue or organ.

The methods of the present invention is provided for an animal, including but not limited to mammals such as a non-primate (e.g., cows, pigs, horses, chickens, cats, dogs, rats, etc.), and a primate (e.g. monkey such as acynomolgous monkey and a human). In a preferred embodiment, the subject is a human.

The present invention is useful alone or in combination with other treatment modalities. In certain embodiments, the treatment of the present invention further includes the administration of one or more immunotherapeutic agents, such as antibodies and immunomodulators, which include, but are not limited to, HERCEPTIN®, RITUXAN®, OVAREX™, PANOREX®, BEC2, IMC-C225, VITAXIN™, CAMPATH® I/H, Smart MI95, LYMPHOCIDE™, Smart I D10, ONCOLYM™, rituximab, gemtuzumab, or trastuzumab. In certain other embodiments, the treatment method further comprises hormonal treatment. Hormonal therapeutic treatments comprise hormonal agonists, hormonal antagonists (e.g., flutamide, tamoxifen, leuprolide acetate (LUPRON™), LH-RH antagonists), inhibitors of hormone biosynthesis and processing, steroids (e.g., dexamethasone, retinoids, betamethasone, cortisol, cortisone, prednisone, dehydrotestosterone, glucocorticoids, mineralocorticoids, estrogen, testosterone, progestins), antigestagens (e.g., mifepristone, onapristone), and antiandrogens (e.g., cyproterone acetate).

5.2.1 Pharmaceutical Compositions

The decellularized extracellular matrix of conditioned body tissue can be formulated into pharmaceutical compositions that are suitable for administration to a subject. Such compositions comprise a prophylactically or therapeutically effective amount of the decellularized extracellular matrix as disclosed herein, and a pharmaceutically acceptable carrier.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete) or, more preferably, MF59C.1 adjuvant available from Chiron, Emeryville, Calif.), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Other examples of suitable pharmaceutical vehicles are described in “Remington: the Science and Practice of Pharmacy”, 20th ed., by Mack Publishing Co. 2000.

Generally, the ingredients of compositions of the invention are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed from an infusion bottle containing sterile pharmaceutical grade water or saline. Where the composition is administered by injection, an ampule of sterile water for injection or saline can be provided so that the ingredients may be mixed prior to administration.

The compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Various delivery systems are known and can be used to administer the compositions of the invention, e.g., encapsulation in liposomes, microparticles, microcapsules, receptor-mediated endocytosis (see, e.g., Wu and Wu, 1987, J. Biol. Chem. 262:4429-4432), etc. Methods of administering a prophylactic or therapeutic amount of the compositions of the invention include, but are not limited to, parenteral administration (e.g., intradermal, intramuscular, intracoronary, intraperitoneal, intravenous and subcutaneous), epidural, and mucosal (e.g., intranasal, inhaled, and oral routes). The composition comprising decellularized extracellular matrix of conditioned body tissue may be administered by any convenient route, for example, by infusion or bolus injection, by absorption through epithelial or mucocutaneous linings (e.g., oral mucosa, rectal and intestinal mucosa, etc.) and may be administered together with other biologically active agents, preferably paclitaxel. Administration can be systemic or local. In addition, it may be desirable to introduce the pharmaceutical composition of the invention into the central nervous system by any suitable route, including intraventricular and intrathecal injection; intraventricular injection may be facilitated by an intraventricular catheter, for example, attached to a reservoir, such as an Ommaya reservoir.

In another embodiment, the decellularized extracellular matrix of conditioned body tissue can be delivered in a controlled release or sustained release system. In one embodiment, a pump may be used to achieve controlled or sustained release (see Langer, 1990, Science 249:1527-1533; Sefton, 1987, CRC Crit. Ref. Biomed. Eng. 14:20; Buchwald et al., 1980, Surgery 88:507; Saudek et al., 1989, N. Engl. J. Med. 321:574). Any technique known to one of skill in the art can be used to produce sustained release formulations comprising the decellularized extracellular matrix of the invention. See, e.g., U.S. Pat. No. 4,526,938; International Publication Nos. WO 91/05548 and WO 96/20698; Ning et al., 1996, Radiotherapy & Oncology 39:179-189; Song et al., 1995, PDA Journal of Pharmaceutical Science & Technology 50:372-397; Cleek et al., 1997, Pro. Int'l. Symp. Control. Rel. Bioact. Mater. 24:853-854; and Lam et al., 1997, Proc. Int'l. Symp. Control Rel. Bioact. Mater. 24:759-760, each of which is incorporated herein by reference in its entirety. In another embodiment, the decellularized extracellular matrix is configured to be controllably released. For example, the decellularized extracellular matrix may be configured to be resorbed by the body of the patient at a predetermined rate. Accordingly, the body of the patient will receive the therapeutic benefits of the decellularized extracellular matrix at the predetermined rate.

In another embodiment, polymeric materials can be used to achieve controlled or sustained release of the decellularized extracellular matrix material (see, e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105); U.S. Pat. Nos. 5,679,377; 5,916,597; 5,912,015; 5,989,463; and 5,128,326; International Publication Nos. WO 99/15154 and WO 99/20253). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glucosides) (PLGA), and polyorthoesters. In a preferred embodiment, the polymer used in a sustained release formulation is inert, free of leachable impurities, stable during storage, sterile, and biodegradable. In yet another embodiment, a controlled or sustained release system can be placed in proximity to the target, thus requiring only a fraction of the systemic dose (see, e.g., Goodson, 1984, in Medical Applications of Controlled Release, supra, vol. 2, pp. 115-138).

The amount of the pharmaceutical composition which will be effective in the treatment of a particular disorder or condition will depend on the nature of the disorder or condition, and can be determined by standard clinical techniques. In addition, in vitro assays and animal models may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses may be extrapolated from dose-response curves derived from in vitro or animal model test systems.

5.2.2 Body Implant

Methods of the present invention also include methods for making and implanting a body implant comprising the decellularized extracellular matrix of body tissue, including conditioned body tissue.

The body implants of the present invention may be, without limitation: (1) vascular implants, such as carotid artery replacement, and general vein and artery replacement in the body; (2) heart valves and patches; (3) burn dressings and coverings; (4) muscle, tooth and bone implants; (5) pericardium and membranes; (6) myocardial patch; (7) urethral sling; and (8) fiber for filling aneurysms.

In one embodiment, the body implant may be a tubular member. The decellularized extracellular matrix is processed into tubular or cylindrical form. The tubular member may be used, for example, to create an esophagus, a vein, an artery, or any other tubular body member. The tubular member may be implanted or placed into a damaged area of a body of a patient. The decellularized extracellular matrix will be resorbed as native or host tissue (tissue produced by the body of the patient) forms. In some cases, the host tissue is functional, vascularized, and morphologically similar to the normal tissue.

In one embodiment of the tubular member, an elastomeric biocompatible polymer is applied to the tubular member. For example, the elastomeric biocompatible polymer may be applied to the tubular member via a spray, glue or other adhesive, radio frequency welding, staples, or any other known method. The elastomeric biocompatible polymer gives the tubular member elasticity and strength.

The elastomeric biocompatible polymer may be a nonporous material or a porous material. For example, the polymer may be a porous material if exchange of fluids and nutrients with the tissues of the patient that surround the implanted tubular member is required. Additionally, the elastomeric biocompatible polymer may be biostable or degradable. For example, a biostable elastomeric biocompatible polymer is used to provide rigidity and elasticity to the tubular member throughout the life of the patient. Examples of the elastomeric biocompatible polymer are silicones (e.g. polysiloxanes and substituted polysiloxanes), polyurethanes, thermoplastic elastomers, ethylene vinyl acetate copolymers, polyolefin elastomers, styrene isobutylene block copolymers, and EPDM rubbers.

In one embodiment, the elastomeric biocompatible polymer is applied to only the outer surface of the tubular member. In another embodiment, the elastomeric biocompatible polymer is applied to only the inner surface of the tubular member. In a further embodiment, the elastomeric biocompatible polymer is applied to both the inner and the outer surfaces of the tubular member.

In one embodiment, the body implant can be an aneurysm treatment device. For example, the aneurysm treatment device may be an aneurysm coil, a seal, a patch, or a filler. In one embodiment, the aneurysm coil is made of the decellularized extracellular matrix. In another embodiment, the aneurysm coil is made of a conventional material and is coated (as described below) with the decellularized extracellular matrix.

The aneurysm treatment device may be placed or implanted into the body of a patient. For example, the aneurysm treatment device may be implanted into the body of a patient as disclosed in U.S. Pat. No. 5,951,599, which is hereby incorporated by reference in its entirety. Once the aneurysm treatment device is placed in the body of the patient, a thrombus will form in the aneurysm. The decellularized extracellular matrix of the aneurysm treatment device will facilitate the formation of functional tissue and will resorb. In one embodiment, the decellularized extracellular matrix is modified to improve the formation of functional tissue. For example, in one embodiment, the decellularized extracellular matrix is coated with a therapeutic agent to improve the formation of functional tissue.

In another embodiment, the body implant is a ventricular restraint such as a sac or a pouch. An example of a cardiac reinforcement or restraint device is disclosed in U.S. Pat. No. 6,544,168 B2, which is hereby incorporated by reference in its entirety. For example, a sheet of the decellularized extracellular matrix may be used to form a sac or a pouch configured to receive a human heart. The sac or pouch includes a cavity that is configured to receive the heart of a patient. In one embodiment, the sac or pouch has a single opening that communicates with the cavity. In another embodiment, the sac or pouch has more than one opening that communicates with the cavity. The sac or pouch may be inserted or implanted into a patient, for example after a myocardial infarction (heart attack), such that the cavity of the sac or pouch receives the heart of the patient. The sac or pouch will provide support to the heart of the patient. Specifically, the sac or pouch will physically prevent the ventricular wall from dilating during tissue remodelling. In one embodiment, the decellularized extracellular matrix will resorb naturally within several months of the implantation or placement into the patient. The mechanical properties of the sac or pouch may be controlled by laminating several sheets of the decellularized extracellular matrix together prior to formation of the sac or pouch. Additionally, the mechanical properties of the sac or pouch may be controlled by perforating the sheet or sheets of the decellularized extracellular matrix to form a mesh.

The modified body implants comprising decellularized extracellular matrix can be implanted in vivo at the site of tissue damage to promote repair, regeneration and/or strengthening. In addition, the materials and methods of this invention are useful to promote the in vitro culture and differentiation of cells and tissues.

5.2.3 Embolotherapy

One aspect of the invention provides for the use of the decellularized extracellular matrix to embolize lesions, tumors, or vessels. In one embodiment, particles of the decellularized extracellular matrix are implanted or injected into a body of a patient to embolize lesions, tumors, or vessels. For example, particles of the decellularized extracellular matrix may be implanted using the embolizing system disclosed in U.S. Pat. No. 5,895,411, which is hereby incorporated by reference in its entirety. As the particles of the decellularized extracellular matrix degrade or resorb, they will be replaced with native tissue, i.e., tissue produced by the body of the patient. In one embodiment, the particles of the decellularized extracellular matrix are suspended in a liquid. In another embodiment, the particles of the decellularized extracellular matrix are placed in the form of a bolus. In yet another embodiment, the decellularized extracellular matrix is mixed with a polymer and formed into spheres. In such an embodiment, the polymer may be biodegradable. Alternatively, the polymer is not biodegradable.

5.2.4 Tissue Regeneration Scaffold

One aspect of the invention provides for the incorporation of the decellularized extracellular matrix of body tissue, including conditioned body tissue, into a biocompatible material for implantation into a subject, preferably human. In a preferred embodiment, the biocompatible material is in the form of a scaffold.

The scaffold may be of natural collagen, decellularized, conditioned extracellular matrix, or synthetic polymer. In certain preferred embodiments, the scaffold serves as a template for cell proliferation and ultimately tissue formation. In a specific embodiment, the scaffold allows the slow release of the decellularized extracellular matrix of the invention into the surrounding tissue. As the cells in the surrounding tissue begin to multiply, they fill up the scaffold and grow into three-dimensional tissue. Blood vessels then attach themselves to the newly grown tissue, the scaffold dissolves, and the newly grown tissue eventually blends in with its surrounding.

5.2.5 Medical Device Comprising Decellularized Extracellular Matrix

The decellularized extracellular matrix of the invention may be used to form medical devices or prosthetic devices, such as a stents, artificial hearts, screws, staples, or clips, which may be implanted in the subject. For example, clips made of the decellularized extracellular matrix of the invention may be used to seal off sections of a stomach of a patient. Alternatively, clips made of the decellularized extracellular matrix of the invention may be used for the treatment of gastroesophageal reflux disease (GERD). In one embodiment, the decellularized extracellular matrix of the invention may be incorporated into the base material needed to make the medical or prosthetic device. For example, in a stent comprising a sidewall of elongated members or wire-like elements, the decellularized extracellular matrix material can be used to form the elongated members or wire-like elements. In another embodiment, the decellularized ECM material of the invention can be used to coat or cover the medical or prosthetic device.

The medical and prosthetic devices of the present invention may be inserted or implanted into the body of a patient.

5.2.5.1 Method of Forming a Medical Device Using Decellularized Extracellular Matrix

In one embodiment, the decellularized extracellular matrix of the invention may be compression molded to form the medical or prosthetic device. Any known method of compression molding may be used. For example, in one embodiment, the decellularized extracellular matrix in a slurry form is poured into a medical or prosthetic device mold. Pressure is then applied to the decellularized extracellular matrix. Once the decellularized extracellular matrix has cured it is removed from the mold. The molded decellularized extracellular matrix may then be inserted or implanted into the body of a patient. In one embodiment, a binder, such as fibrin glue, is mixed or otherwise incorporated with the decellularized matrix. In another embodiment, the decellularized extracellular matrix or the collagen of the matrix is cross-linked, such as via ultra-violet light or extreme dehydration, after the compression of the material. In yet another embodiment, the collagen of the decellularized extracellular matrix is reversibly denatured during the compression stage and is then reversed after the compression stage. For example, the collagen may have a pH of less than 4 during compression and a pH of about 7.4 after compression.

5.2.5.2 Types of Medical Devices for Coating

Medical devices that are useful in the present invention can be made of any biocompatible material suitable for medical devices in general which include without limitation natural polymers, synthetic polymers, ceramics and metallics. Metallic material is more preferable. Suitable metallic materials include metals and alloys based on titanium (such as nitinol, nickel titanium alloys, thermo-memory alloy materials), stainless steel, tantalum, nickel-chrome, or certain cobalt alloys including cobalt-chromium-nickel alloys such as Elgiloy® and Phynox®. Metallic materials also include clad composite filaments, such as those disclosed in WO 94/16646.

Metallic materials may be made into elongated members or wire-like elements and then woven to form a network of metal mesh. Polymer filaments may also be used together with the metallic elongated members or wire-like elements to form a network mesh. If the network is made of metal, the intersection may be welded, twisted, bent, glued, tied (with suture), heat sealed to one another; or connected in any manner known in the art.

The polymer(s) useful for forming the medical device should be ones that are biocompatible and avoid irritation to body tissue. They can be either biostable or bioabsorbable. Suitable polymeric materials include without limitation polyurethane and its copolymers, silicone and its copolymers, ethylene vinyl-acetate, polyethylene terephtalate, thermoplastic elastomers, polyvinyl chloride, polyolefins, cellulosics, polyamides, polyesters, polysulfones, polytetrafluorethylenes, polycarbonates, acrylonitrile butadiene styrene copolymers, acrylics, polylactic acid, polyglycolic acid, polycaprolactone, polylactic acid-polyethylene oxide copolymers, cellulose, collagens, and chitins.

Other polymers that are useful as materials for medical devices include without limitation dacron polyester, poly(ethylene terephthalate), polycarbonate, polymethylmethacrylate, polypropylene, polyalkylene oxalates, polyvinylchloride, polyurethanes, polysiloxanes, nylons, poly(dimethyl siloxane), polycyanoacrylates, polyphosphazenes, poly(amino acids), ethylene glycol I dimethacrylate, poly(methyl methacrylate), poly(2-hydroxyethyl methacrylate), polytetrafluoroethylene poly(HEMA), polyhydroxyalkanoates, polytetrafluorethylene, polycarbonate, poly(glycolide-lactide) co-polymer, polylactic acid, poly(ε-caprolactone), poly(β-hydroxybutyrate), polydioxanone, poly(γ-ethyl glutamate), polyiminocarbonates, poly(ortho ester), polyanhydrides, alginate, dextran, chitin, cotton, polyglycolic acid, polyurethane, or derivatized versions thereof, i.e., polymers which have been modified to include, for example, attachment sites or cross-linking groups, e.g., RGD, in which the polymers retain their structural integrity while allowing for attachment of molecules, such as proteins, nucleic acids, and the like.

Furthermore, although the invention can be practiced by using a single type of polymer to form the medical device, various combinations of polymers can be employed. The appropriate mixture of polymers can be coordinated to produce desired effects when incorporated into a medical device. In certain preferred embodiments, the decellularized extracellular matrix is mixed with a polymer.

The decellularized extracellular matrix of the invention may also be used alone or in combination with a polymer described above to form the medical device. The decellularized extracellular matrix may be dried to increase its mechanical strength. The dried decellularized extracellular matrix may then be used as the base material to form a whole or part of the medical device. In preferred embodiments, the decellularized extracellular matrix constitutes at least 5%, at least 10%, at least 25%, at least 50%, at least 80%, at least 90%, at least 95%, at least 99% by weight or by size of the medical device.

Examples of the medical devices suitable for the present invention include, but are not limited to, stents, surgical staples, catheters (e.g., central venous catheters and arterial catheters), guidewires, cannulas, cardiac pacemaker leads or lead tips, cardiac defibrillator leads or lead tips, implantable vascular access ports, blood storage bags, blood tubing, vascular or other grafts, intra-aortic balloon pumps, heart valves, cardiovascular sutures, total artificial hearts and ventricular assist pumps, and extra-corporeal devices such as blood oxygenators, blood filters, hemodialysis units, hemoperfusion units and plasmapheresis units.

Medical devices of the present invention include those that have a tubular or cylindrical-like portion. The tubular portion of the medical device need not to be completely cylindrical. For instance, the cross-section of the tubular portion can be any shape, such as rectangle, a triangle, etc., not just a circle. Such devices include, without limitation, stents and grafts. A bifurcated stent is also included among the medical devices which can be fabricated by the method of the present invention.

Medical devices which are particularly suitable for the present invention include any kind of stent for medical purposes which is known to the skilled artisan. Suitable stents include, for example, vascular stents such as self-expanding stents and balloon expandable stents. Examples of self-expanding stents useful in the present invention are illustrated in U.S. Pat. Nos. 4,655,771 and 4,954,126 issued to Wallsten and 5,061,275 issued to Wallsten et al. Examples of appropriate balloon-expandable stents are shown in U.S. Pat. No. 5,449,373 issued to Pinchasik et al.

5.2.5.3 Methods of Coating the Medical Device

In the present invention, the decellularized extracellular matrix of the invention, preferably in combination with a biologically active material such as paclitaxel, can be applied by any method to a surface of a medical device to form a coating. Examples of suitable methods are spraying, laminating, pressing, brushing, swabbing, dipping, rolling, electrostatic deposition and all modern chemical ways of immobilization of bio-molecules to surfaces. Preferably, the decellularized extracellular matrix is applied to a surface of a medical device by spraying, rolling, laminating, and pressing. In one embodiment of the present invention, more than one coating method can be used to make a medical device. In certain embodiments, the decellularized extracellular matrix is placed into a carrier in order to apply it to the device surface. Non-limiting examples of carriers include SIBS, PLGA, PGA, collagen (all types), etc.

Furthermore, before applying the coating composition, the surface of the medical device is optionally subjected to a pre-treatment, such as roughening, oxidizing, sputtering, plasma-deposition or priming in embodiments where the surface to be coated does not comprise depressions. Sputtering is a deposition of atoms on the surface by removing the atom from the cathode by positive ion bombardment through a gas discharge. Also, exposing the surface of the device to a primer is a possible method of pre-treatment.

Coating compositions suitable for applying coating materials to the devices of the present invention can include a polymeric material and preferably a biologically active material dispersed or dissolved in a solvent suitable for the medical device, which are known to the skilled artisan. The solvents used to prepare coating compositions include ones which can dissolve the polymeric material into solution or suspend the polymeric material and do not alter or adversely impact the therapeutic properties of the biologically active material employed. For example, useful solvents for silicone include tetrahydrofuran (THF), chloroform, toluene, acetone, isooctane, 1,1,1-trichloroethane, dichloromethane, and mixture thereof.

The polymeric material should be a material that is biocompatible and avoids irritation to body tissue. Preferably the polymeric materials used in the coating composition of the present invention are selected from the following: polyurethanes, silicones (e.g., polysiloxanes and substituted polysiloxanes), and polyesters. Also preferable as a polymeric material is styrene-isobutylene-styrene (SIBS). Other polymers which can be used include ones that can be dissolved and cured or polymerized on the medical device or polymers having relatively low melting points that can be blended with biologically active materials. Additional suitable polymers include, thermoplastic elastomers in general, polyolefins, polyisobutylene, ethylene-alphaolefin copolymers, acrylic polymers and copolymers, vinyl halide polymers and copolymers such as polyvinyl chloride, polyvinyl ethers such as polyvinyl methyl ether, polyvinylidene halides such as polyvinylidene fluoride and polyvinylidene chloride, polyacrylonitrile, polyvinyl ketones, polyvinyl aromatics such as polystyrene, polyvinyl esters such as polyvinyl acetate, copolymers of vinyl monomers, copolymers of vinyl monomers and olefins such as ethylene-methyl methacrylate copolymers, acrylonitrile-styrene copolymers, ABS (acrylonitrile-butadiene-styrene) resins, ethylene-vinyl acetate copolymers, polyamides such as Nylon 66 and polycaprolactone, alkyd resins, polycarbonates, polyoxymethylenes, polyimides, polyethers, epoxy resins, rayon-triacetate, cellulose, cellulose acetate, cellulose butyrate, cellulose acetate butyrate, cellophane, cellulose nitrate, cellulose propionate, cellulose ethers, carboxymethyl cellulose, collagens, chitins, polylactic acid, polyglycolic acid, polylactic acid-polyethylene oxide copolymers, EPDM (etylene-propylene-diene) rubbers, fluorosilicones, polyethylene glycol, polysaccharides, phospholipids, and combinations of the foregoing.

More preferably for medical devices which undergo mechanical challenges, e.g. expansion and contraction, the polymeric materials should be selected from elastomeric polymers such as silicones (e.g. polysiloxanes and substituted polysiloxanes), polyurethanes, thermoplastic elastomers, ethylene vinyl acetate copolymers, polyolefin elastomers, and EPDM rubbers. Because of the elastic nature of these polymers, the coating composition is capable of undergoing deformation under the yield point when the device is subjected to forces, stress or mechanical challenge.

The term “biologically active material” encompasses therapeutic agents, such as drugs, and also genetic materials and biological materials. The genetic materials mean DNA or RNA, including, without limitation, of DNA/RNA encoding a useful protein stated below, intended to be inserted into a human body including viral vectors and non-viral vectors. The biological materials include cells, yeasts, bacteria, proteins, peptides, cytokines and hormones. Examples for peptides and proteins include vascular endothelial growth factor (VEGF), transforming growth factor (TGF), fibroblast growth factor (FGF), epidermal growth factor (EGF), cartilage growth factor (CGF), nerve growth factor (NGF), keratinocyte growth factor (KGF), skeletal growth factor (SGF), osteoblast-derived growth factor (BDGF), hepatocyte growth factor (HGF), insulin-like growth factor (IGF), cytokine growth factors (CGF), platelet-derived growth factor (PDGF), hypoxia inducible factor-1 (HIF-1), stem cell derived factor (SDF), stem cell factor (SCF), endothelial cell growth supplement (ECGS), granulocyte macrophage colony stimulating factor (GM-CSF), growth differentiation factor (GDF), integrin modulating factor (IMF), calmodulin (CaM), thymidine kinase (TK), tumor necrosis factor (TNF), growth hormone (GH), bone morphogenic protein (BMP) (e.g., BMP-2, BMP-3, BMP-4, BMP-5, BMP-6 (Vgr-1), BMP-7 (PO-1), BMP-8, BMP-9, BMP-10, BMP-11, BMP-12, BMP-14, BMP-15, BMP-16, etc.), matrix metalloproteinase (MMP), tissue inhibitor of matrix metalloproteinase (TIMP), cytokines, interleukin (e.g., IL-1, IL-2, IL-3, IL-4, IL-5, IL-6, IL-7, IL-8, IL-9, IL-10, IL-11, IL-12, IL-15, etc.), lymphokines, interferon, integrin, collagen (all types), elastin, fibrillins, fibronectin, vitronectin, laminin, glycosaminoglycans, proteoglycans, transferrin, cytotactin, cell binding domains (e.g., RGD), and tenascin. Currently preferred BMP's are BMP-2, BMP-3, BMP-4, BMP-5, BMP-6, BMP-7. These dimeric proteins can be provided as homodimers, heterodimers, or combinations thereof, alone or together with other molecules. Cells can be of human origin (autologous or allogeneic) or from an animal source (xenogeneic), genetically engineered, if desired, to deliver proteins of interest at the transplant site. The delivery media can be formulated as needed to maintain cell function and viability. Cells include progenitor cells (e.g., endothelial progenitor cells), stem cells (e.g., mesenchymal, hematopoietic, neuronal), stromal cells, parenchymal cells, undifferentiated cells, fibroblasts, macrophage, and satellite cells. Biologically active materials also include non-genetic therapeutic agents, such as:

anti-thrombogenic agents such as heparin, heparin derivatives, urokinase, and PPack (dextrophenylalanine proline arginine chloromethylketone);

anti-proliferative agents such as enoxaprin, angiopeptin, or monoclonal antibodies capable of blocking smooth muscle cell proliferation, hirudin, and acetylsalicylic acid, amlodipine and doxazosin;

anti-inflammatory agents such as glucocorticoids, betamethasone, dexamethasone, prednisolone, corticosterone, budesonide, estrogen, sulfasalazine, and mesalamine;

antineoplastic/antiproliferative/anti-mitotic agents such as paclitaxel, 5-fluorouracil, cisplatin, vinblastine, cladribine, vincristine, epothilones, methotrexate, azathioprine, adriamycin and mutamycin; endostatin, angiostatin and thymidine kinase inhibitors, taxol and its analogs or derivatives;

anesthetic agents such as lidocaine, bupivacaine, and ropivacaine;

anti-coagulants such as D-Phe-Pro-Arg chloromethyl keton, an RGD peptide-containing compound, heparin, antithrombin compounds, platelet receptor antagonists, anti-thrombin antibodies, anti-platelet receptor antibodies, aspirin (aspirin is also classified as an analgesic, antipyretic and anti-inflammatory drug), dipyridamole, protamine, hirudin, prostaglandin inhibitors, platelet inhibitors and tick antiplatelet peptides;

DNA demethylating drugs such as 5-azacytidine, which is also categorized as a RNA or DNA metabolite that inhibit cell growth and induce apoptosis in certain cancer cells;

vascular cell growth promoters such as growth factors, vascular endothelial growth factors (VEGF, all types including VEGF-2), growth factor receptors, transcriptional activators, and translational promoters;

vascular cell growth inhibitors such as antiproliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin;

cholesterol-lowering agents; vasodilating agents; and agents which interfere with endogenous vasoactive mechanisms;

anti-oxidants, such as probucol;

antibiotic agents, such as penicillin, cefoxitin, oxacillin, tobranycin, rapamycin;

angiogenic substances, such as acidic and basic fibroblast growth factors, estrogen including estradiol (E2), estriol (E3) and 17-Beta Estradiol; and

drugs for heart failure, such as digoxin, beta-blockers, angiotensin-converting enzyme (ACE) inhibitors including captopril, enalopril, and statins and related compounds.

In certain embodiments, the medical device of the present invention is covered with one coating layer. In certain other embodiments, the medical device of the present invention is covered with more than one coating layer. In preferred embodiments, the medical device is covered with different coating layers. For example, the coating can comprise a first layer and a second layer that contain different biologically active materials. Alternatively, the first layer and the second layer may contain an identical biologically active material having different concentrations. In one embodiment, either the first layer or the second layer may be free of biologically active material.

### 6. EXAMPLES

6.1 Increased Vega Levels in Submucosa

Porcine intestines are conditioned with plasmid DNA encoding human VEGF. The DNA is delivered using a drug delivery balloon (Remedy, Boston Scientific, Natick, Mass.) which is placed in the intestine. Following infusion, the animal is allowed to live normally for one week. After this time, the animal is sacrificed and the targeted region of the intestine is isolated. Finally, the submucosal layer is isolated from the muscular layers and further processed to remove cells. The transfection with DNA results in higher levels of VEGF in the tissue, and hence an improved tissue regeneration scaffold. Ultrasound or iontophoresis may be used to improve conditioning of the tissue. These techniques are used during delivery of the DNA to enhance diffusion into the tissue and potentially increase transfection by disrupting cell membranes.

6.2 Increased Stem Cell Derived Factor (SDF) in Submucosa

Small intestine submucosa is being tested as a material for aortic-valve leaflet replacement. Small pieces of the isolated submucosa are attached to a valve annulus. The new valve is implanted and functions normally as cells from the blood stream migrate into and populate the submucosa. These cells ultimately form a new valve by replacing the transplanted leaflets. However, this process is slow and not always complete. In this example, submucosa is modified to contain additional basic fibroblast growth factor (bFGF) or stem cell derived factor (SDF).

The artery and vein supplying blood to a section of the small intestine is isolated. Identification of the vessels and affected region of the intestine is aided through the use of radiography and x-ray contrast media. Additionally, the area of the intestine served by the artery can be highlighted by a blanching when blood flow is interrupted. A solution of adenovirus encoding bFGF or SDF is prepared. The target vein and arteries are clamped and a sufficient volume of the solution is infused into either the artery or vein. A blanching of the target section of the intestine may be observed. To facilitate infusion, the clamps may be loosened until a sufficient volume of the solution can be delivered. Pressurization of the target section using the delivery syringe or hanging bag may be used. Following delivery, the clamps are removed and the animal is sacrificed 5-10 days later. The submucosa layer is prepared as described above. Sheets of submucosa can then be turned into valves using established techniques.

6.3 Vega Enhanced Bone Marrow Extracellular Matrix

Bone marrow extracellular matrix has potential therapeutic value in treating myocardial infarction. Increasing the amount of TGF-beta in the matrix will provide additional benefit. In this example, a solution containing plasmid encoding TGF-beta is injected into the marrow cavity of the sternum. A needle attached to a syringe is pushed through the cortical bone and into the marrow space. About 1 ml of the solution is injected. This procedure is repeated up to 15 times. The animal is sacrificed one week later and the sternum processed to isolate the bone marrow ECM.

### 7. Equivalents

The present invention is not to be limited in scope by the specific embodiments described which are intended as single illustrations of individual aspects of the invention, and functionally equivalent methods and components are within the scope of the invention. Indeed, various modifications of the invention, in addition to those shown and described herein, will become apparent to those skilled in the art from the foregoing description and accompanying drawings using no more than routine experimentation. Such modifications and equivalents are intended to fall within the scope of the appended claims.

All publications, patents and patent applications mentioned in this specification are herein incorporated by reference into the specification to the same extent as if each individual publication, patent or patent application was specifically and individually indicated to be incorporated herein by reference.

Citation or discussion of a reference herein shall not be construed as an admission that such is prior art to the present invention.

1. An apparatus, comprising:
a medical device having a biodegradable material and a decellularized extracellular matrix,
wherein the medical device is configured to be implanted into a human patient, the biodegradable material is formulated to degrade within the human patient, the decellularized extracellular matrix is formulated to be released as the biodegradable material degrades.

2. The apparatus of claim 1, wherein the biodegradable material is a biodegradable polymer.

3. The apparatus of claim 1, wherein the medical device is configured to be injected into a human patient.

4. An apparatus, comprising:
a compression molded medical device, the compression molded medical device including a decellularized extracellular matrix.

5. The apparatus of claim 4, wherein the medical device is a screw.

6. The apparatus of claim 4, wherein the medical device is a staple.

7. The apparatus of claim 4, wherein the medical device is a clip.

8. The apparatus of claim 4, wherein the medical device is configured to be implanted into a human patient.

9. An apparatus, comprising:
a medical device defining a cavity, the medical device including a decellularized extracellular matrix, the cavity being configured to receive a heart of a patient.

10. The apparatus of claim 9, wherein the medical device defines an opening that communicates with the cavity.

11. A method making a medical device, comprising:
providing a decellularized extracellular matrix; and
forming a pouch that defines a cavity, the cavity being configured to receive a heart of a mammal.

12. An apparatus, comprising:
a tubular member having a decellularized extracellular matrix layer and a elastomeric biocompatible polymer layer, the tubular member being configured to be implanted into a body of a mammal.

13. The apparatus of claim 12, wherein the elastomeric biocompatible polymer layer is a porous material.

14. The apparatus of claim 12, wherein the elastomeric biocompatible polymer layer is a nonporous material.

15. The apparatus of claim 12, wherein the elastomeric biocompatible polymer layer is disposed on an outer surface of the tubular member.

16. The apparatus of claim 12, wherein the elastomeric biocompatible polymer layer is disposed on an inner surface of the tubular member.

17. The apparatus of claim 12, wherein the elastomeric biocompatible polymer layer is a first elastomeric biocompatible polymer layer, the tubular member has a second elastomeric biocompatible polymer layer, the first elastomeric biocompatible polymer layer being disposed on an outer surface of the tubular member, the second elastomeric biocompatible polymer layer being disposed on an inner surface of the tubular member.

18. The apparatus of claim 12, wherein the tubular member is configured to be implanted onto the body of a mammal.
